(19) United States
(12) Patent Application Publication
(10) Pub. No.: US 2023/0365540 A1
(43) Pub. Date: Nov. 16, 2023

(54) SMALL MOLECULE INHIBITORS OF ENPP1

(71) Applicant: The Regents of the University of California, Oakland, CA (US)

(72) Inventors: Arjun Deb, Los Angeles, CA (US); Michael E. Jung, Los Angeles, CA (US); Xiaohong Chen, Los Angeles, CA (US); Shen Li, Los Angeles, CA (US); Yanpeng Xing, Los Angeles, CA (US); Hui Ding, Los Angeles, CA (US)

(21) Appl. No.: 18/025,487

(22) PCT Filed: Sep. 9, 2021
(86) PCT No.: PCT/US21/49573

§ 371 Date: Mar. 9, 2023

Related U.S. Application Data
(60) Provisional application No. 63/076,137, filed on Sep. 9, 2020.

(51) Int. Cl. C07D 405/12; C07D 409/12; C07D 413/14; C07D 417/14; C07D 215/227; C07D 409/14; C07D 405/14; C07C 275/30; C07C 275/32; C07C 237/30; C07D 231/14

(52) U.S. Cl. CPC C07D 405/12; C07D 409/12; C07D 413/14; C07D 417/14; C07D 215/227; C07D 409/14; C07D 405/14; C07C 275/30; C07C 275/32; C07C 237/30; C07D 231/14

(57) ABSTRACT

The present disclosure relates to compounds that are capable of inhibiting the ENPP1 gene and treating a variety of cardiac conditions. The disclosure further relates to methods of treating or preventing cardiac conditions, such as myocardial infarction and heart failure.

### RELATED APPLICATION

This application claims the benefit of U.S. Provisional Application No. 63/076,137, filed Sep. 9, 2020, the contents of which is fully incorporated by reference herein.

### STATEMENT REGARDING FEDERALLY SPONSORED RESEARCH

This invention was made with government support under Grant Numbers HL137241, HL119893 and AR075867, awarded by the National Institutes of Health, and Grant Number W81XWH-17-1-0464 and W81XWH-20-1-0238 awarded by U.S. Department of Defense. The government has certain rights in the invention.

### BACKGROUND

After acute ischemic injury, the heart regenerates dead cardiac muscle poorly and lost heart muscle is replaced by non-contractile scar tissue. Such scar tissue increases the hemodynamic burden on the remaining cardiac muscle and, over time, ventricles will often fail, leading to a cycle of ventricular dilatation, worsening fibrosis and progressive decline in cardiac function. Scar tissue is an independent predictor of mortality and cardiovascular outcomes after heart injury. More than 700,000 patients are annually diagnosed with heart failure, and more than 40% of these cases are the result of heart attack or myocardial infarction. Thus, modulation of cardiac wound healing to redirect the cardiac injury response from a fibrotic to a reparative one with minimal adverse remodeling and decline in heart function is an unmet need of cardiovascular therapeutics.

### SUMMARY OF THE INVENTION

In certain aspects, the present disclosure provides compounds of Formulas I, II, III, or IV:

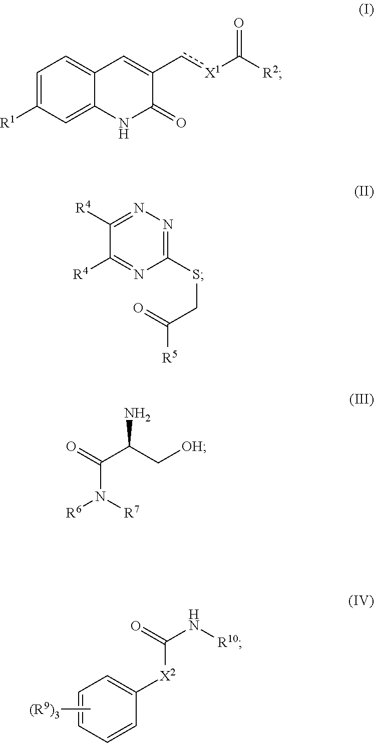

and pharmaceutically acceptable salts thereof;
wherein:
R¹ is selected from —OH, alkoxy, or halo;
R² is selected from alkyl, aryl, or heteroaryl;
X¹ is —N(R³)— and [symbol] is a single bond or X is ═C(H)— and [symbol] is a double bond;
R³ is aralkyl or heteroaralkyl;
R⁴ is heteroaryl;
R⁵ is selected from arylamine or aryl;
R⁶ is H;
R⁷ is selected from

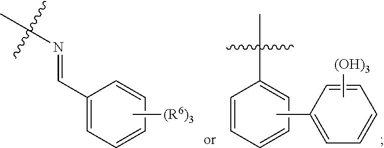

or R⁶ and R⁷, together with the N to which they are attached, come together to form

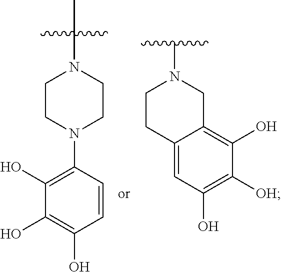

and
R⁸ is, independently for each occurrence, a non-hydrogen substituent, e.g., a substituent selected from —OH, —NHAc, halo, or -alkylene-OH;
R⁹ is, independently for each occurrence, a non-hydrogen substituent, e.g., a substituent selected from —OH or alkoxy;
X² is selected from alkylene, alkylene-NH—, or heteroarylene; and
R¹⁰ is selected from -alkylene-C(O)NHOH, -alkylene-C(O)NH-alkoxy, -alkylene-C(O)NH-alkyl, aryl, or -alkylene-aryl, wherein aryl is optionally substituted, e.g., with 1-3 substituents selected, independently for each occurrence, from halo, —OH, or alkoxy.

In certain aspects, the present disclosure provides pharmaceutical compositions comprising compounds of the present disclosure, or pharmaceutically acceptable salts thereof, together with one or more pharmaceutically acceptable excipients, diluents, or carriers.

In certain aspects, the present disclosure provides methods of treating myocardial infarction, preventing heart failure, promoting cardiac wound healing, preventing ectopic calcification of cardiac tissue, preventing scarring of cardiac tissue, preventing dilated cardiomyopathy, enhancing cardiac repair, preventing cell death of cardiac cells, preventing release of one or more pro-inflammatory molecules from cardiac myocytes, and/or inhibiting ENPP1 activity in a subject in need thereof, the method comprising administering to the subject a therapeutically acceptable amount of a compound or pharmaceutical composition of the present disclosure.

### DETAILED DESCRIPTION OF THE INVENTION

In certain aspects, the present disclosure provides compounds of Formulas I, II, III, or IV:

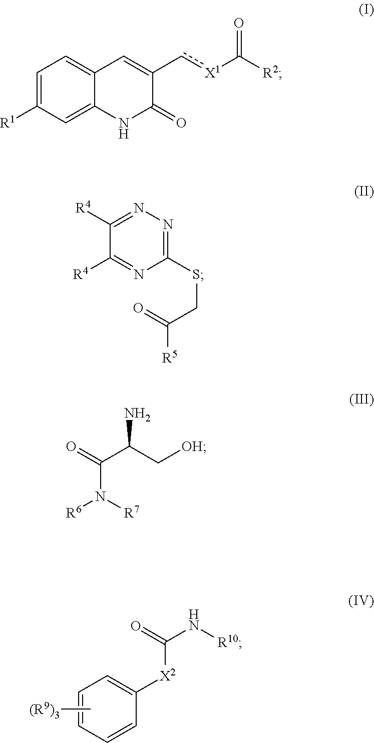

and pharmaceutically acceptable salts thereof;
wherein:
R¹ is selected from —OH, alkoxy, or halo;
R² is selected from alkyl, aryl, or heteroaryl;
X¹ is —N(R³)— and [symbol] is a single bond or X is ═C(H)— and [symbol] is a double bond;
R³ is aralkyl or heteroaralkyl;
R⁴ is heteroaryl;
R⁵ is selected from arylamine or aryl;
R⁶ is H;
R⁷ is selected from

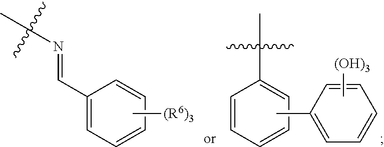

or R⁶ and R⁷, together with the N to which they are attached, come together to form

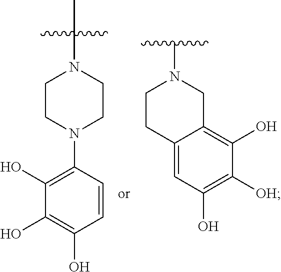

and
R⁸ is, independently for each occurrence, a non-hydrogen substituent, e.g., a substituent selected from —OH, —NHAc, halo, or -alkylene-OH;
R⁹ is, independently for each occurrence, a non-hydrogen substituent, e.g., a substituent selected from —OH or alkoxy;
X² is selected from alkylene, alkylene-NH—, or heteroarylene; and R¹⁰ is selected from -alkylene-C(O)NHOH, -alkylene-C(O)NH-alkoxy, -alkylene-C(O)NH-alkyl, aryl, or -alkylene-aryl, wherein aryl is optionally substituted, e.g., with 1-3 substituents selected, independently for each occurrence, from halo, —OH, or alkoxy.

In certain embodiments, the compound is a compound of Formula I, for example:

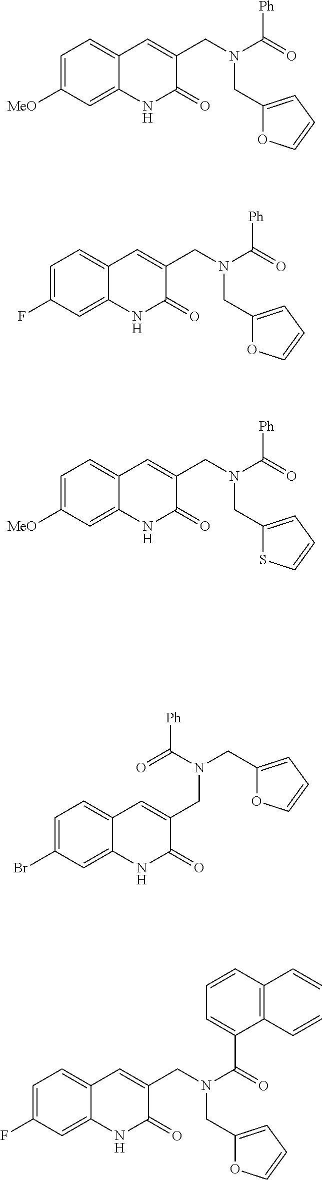 [SMILES: COc1ccc2cc(CN(Cc3ccco3)C(=O)c3ccccc3)c(=O)[nH]c2c1 ; O=C(c1ccccc1)N(Cc1ccco1)Cc1cc2ccc(F)cc2[nH]c1=O ; COc1ccc2cc(CN(Cc3cccs3)C(=O)c3ccccc3)c(=O)[nH]c2c1 ; O=C(c1ccccc1)N(Cc1ccco1)Cc1cc2ccc(Br)cc2[nH]c1=O ; C=Cc1cccc(C(=O)N(Cc2ccco2)Cc2cc3ccc(F)cc3[nH]c2=O)c1C=C]

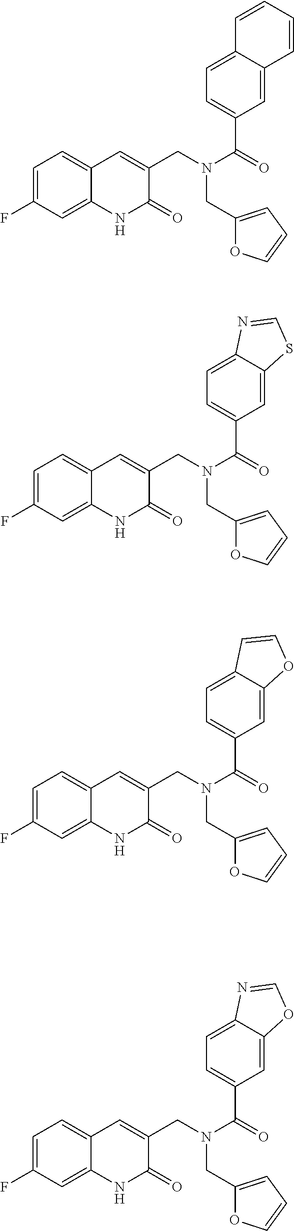 [SMILES: O=C(c1ccc2ccccc2c1)N(Cc1ccco1)Cc1cc2ccc(F)cc2[nH]c1=O ; O=C(c1ccc2ncsc2c1)N(Cc1ccco1)Cc1cc2ccc(F)cc2[nH]c1=O ; O=C(c1ccc2ccoc2c1)N(Cc1ccco1)Cc1cc2ccc(F)cc2[nH]c1=O ; O=C(c1ccc2ncoc2c1)N(Cc1ccco1)Cc1cc2ccc(F)cc2[nH]c1=O]

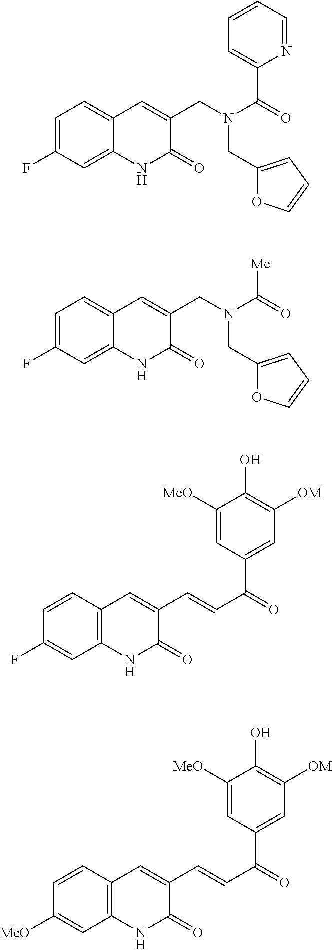

or a pharmaceutically acceptable salt thereof.

In certain embodiments, the compound is a compound of Formula II, for example:

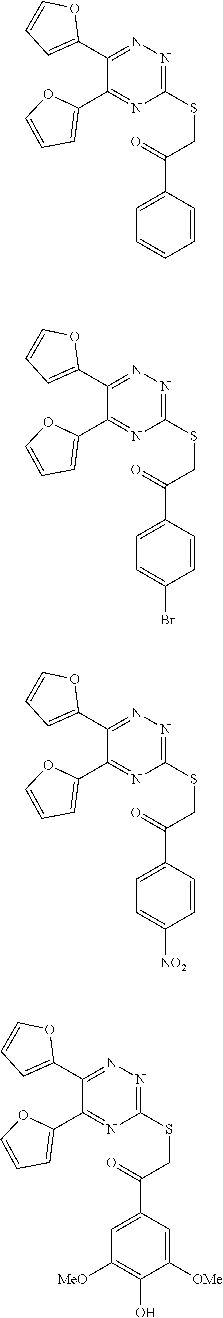 [SMILES: O=C(CSc1nnc(-c2ccco2)c(-c2ccco2)n1)c1ccccc1 ; O=C(CSc1nnc(-c2ccco2)c(-c2ccco2)n1)c1ccc(Br)cc1 ; O=C(CSc1nnc(-c2ccco2)c(-c2ccco2)n1)c1ccc([N+](=O)[O-])cc1 ; COc1cc(C(=O)CSc2nnc(-c3ccco3)c(-c3ccco3)n2)cc(OC)c1O]

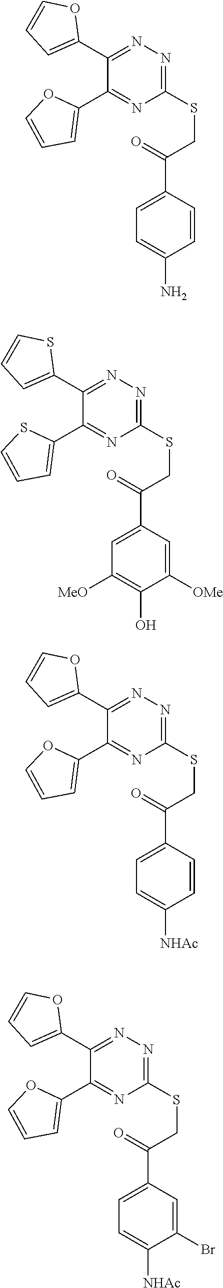 [SMILES: Nc1ccc(C(=O)CSc2nnc(-c3ccco3)c(-c3ccco3)n2)cc1 ; COc1cc(C(=O)CSc2nnc(-c3cccs3)c(-c3cccs3)n2)cc(OC)c1O ; CC(=O)Nc1ccc(C(=O)CSc2nnc(-c3ccco3)c(-c3ccco3)n2)cc1 ; CC(=O)Nc1ccc(C(=O)CSc2nnc(-c3ccco3)c(-c3ccco3)n2)cc1Br]

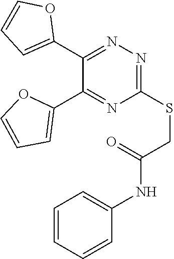 [SMILES: O=C(CSc1nnc(-c2ccco2)c(-c2ccco2)n1)Nc1ccccc1]

or a pharmaceutically acceptable salt thereof.

In certain embodiments, the compound is a compound of Formula III, for example:

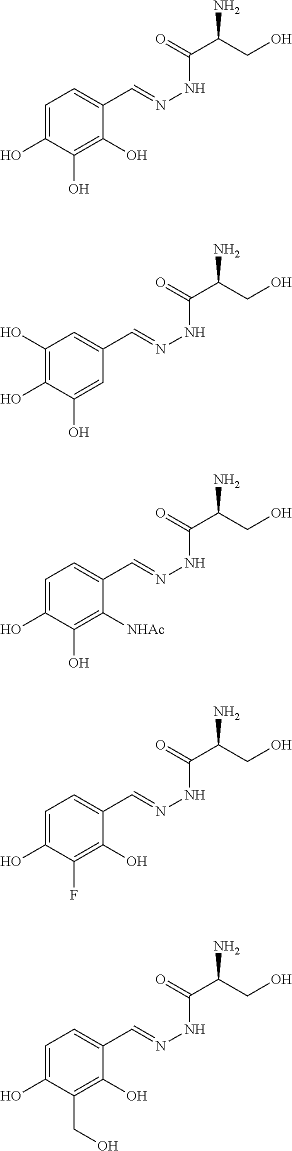 [SMILES: N[C@@H](CO)C(=O)N/N=C/c1ccc(O)c(O)c1O ; N[C@@H](CO)C(=O)N/N=C/c1cc(O)c(O)c(O)c1 ; CC(=O)Nc1c(/C=N/NC(=O)[C@@H](N)CO)ccc(O)c1O ; N[C@@H](CO)C(=O)N/N=C/c1ccc(O)c(F)c1O ; N[C@@H](CO)C(=O)N/N=C/c1ccc(O)c(CO)c1O]

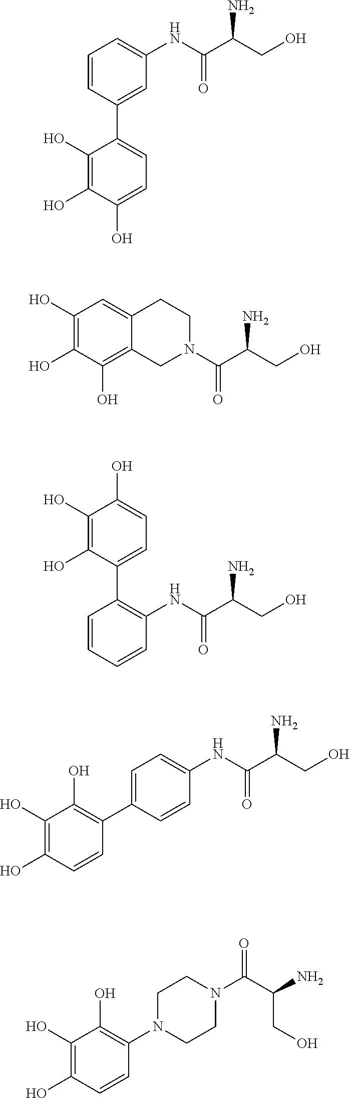 [SMILES: N[C@@H](CO)C(=O)Nc1cccc(-c2ccc(O)c(O)c2O)c1 ; N[C@@H](CO)C(=O)N1CCc2cc(O)c(O)c(O)c2C1 ; N[C@@H](CO)C(=O)Nc1ccccc1-c1ccc(O)c(O)c1O ; N[C@@H](CO)C(=O)Nc1ccc(-c2ccc(O)c(O)c2O)cc1 ; N[C@@H](CO)C(=O)N1CCN(c2ccc(O)c(O)c2O)CC1]

or a pharmaceutically acceptable salt thereof.

In certain embodiments, the compound is a compound of Formula IV, for example:

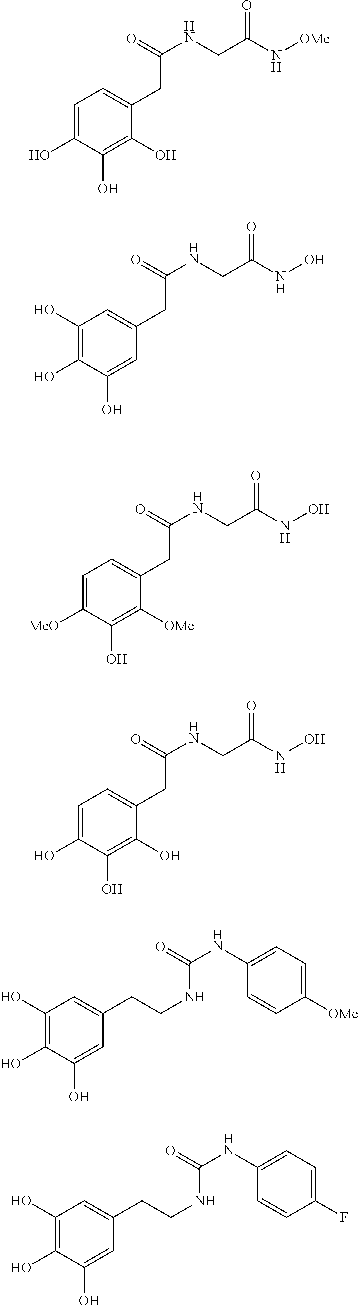 [SMILES: CONC(=O)CNC(=O)Cc1ccc(O)c(O)c1O ; O=C(CNC(=O)Cc1cc(O)c(O)c(O)c1)NO ; COc1ccc(CC(=O)NCC(=O)NO)c(OC)c1O ; O=C(CNC(=O)Cc1ccc(O)c(O)c1O)NO ; COc1ccc(NC(=O)NCCc2cc(O)c(O)c(O)c2)cc1 ; O=C(NCCc1cc(O)c(O)c(O)c1)Nc1ccc(F)cc1]

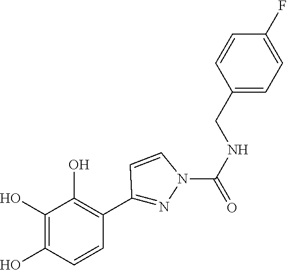 [SMILES: O=C(NCc1ccc(F)cc1)n1ccc(-c2ccc(O)c(O)c2O)n1]

or a pharmaceutically acceptable salt thereof.

In certain aspects, the present disclosure provides pharmaceutical compositions comprising compounds of the present disclosure, or pharmaceutically acceptable salts thereof, together with one or more pharmaceutically acceptable excipients, diluents, or carriers.

In certain aspects, the present disclosure provides methods of treating myocardial infarction, preventing heart failure, promoting cardiac wound healing, preventing ectopic calcification of cardiac tissue, preventing scarring of cardiac tissue, preventing dilated cardiomyopathy, enhancing cardiac repair, preventing cell death of cardiac cells, preventing release of one or more pro-inflammatory molecules from cardiac myocytes, and/or inhibiting ENPP1 activity in a subject in need thereof, the method comprising administering to the subject a therapeutically acceptable amount of a compound or pharmaceutical composition of the present disclosure.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃-30 for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

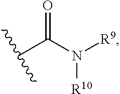

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

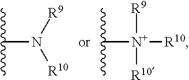

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

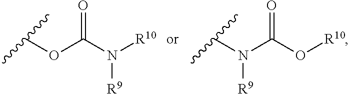

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

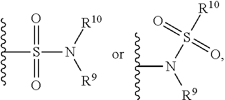

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

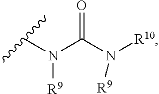

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention, and are not intended to limit the invention.

### Example 1: Preparation and Characterization of Exemplary Compounds

### Synthesis of N-(Furan-2-ylmethyl)-N-((7-methoxy-2-oxo-1,2-dihydroquinolin-3-yl)methyl)benzamide (JXC010)

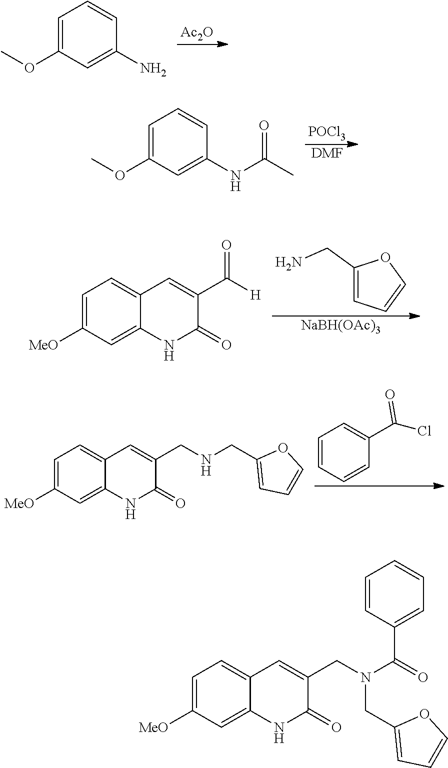

A mixture of 3-methoxyaniline (1.8 mL, 16.2 mmol) and acetic anhydride (2.0 mL, 21.2 mmol) in dioxane 20 mL was heated at 60° C. for 2 h. The reaction mixture was cooled to room temperature, concentrated in vacuo, and the residue was recrystallized from 80% aqueous EtOH, then dried in vacuo to provide N-(3-methoxyphenyl)acetamide as a white crystalline powder in 85% yield.

A solution of the N-(3-methoxyphenyl)acetamide (1.6 g, 10 mmol) and phosphorus oxychloride (0.65 mL, 7 mmol) in DMF 6.0 mL was heated at 80° C. for 20 h and cooled to room temperature. The mixture was poured onto ice (30 g), and solid Na₂CO₃ (10 g) was added with stirring. After standing for 1 h, a brownish precipitate formed, which was filtered, washed with water and MeOH, and recrystallized from EtOAc. The solids were filtered and dried in vacuo to provide 7-methoxy-2-oxo-1,2-dihydroquinoline-3-carboxaldehyde as a yellow solid in 65% yield.

A mixture of the 7-methoxy-2-oxo-1,2-dihydroquinoline-3-carboxaldehyde (1.0 g, 5.0 mmol), furan-2-ylmethanamine (275 mg, 5.5 mmol) and sodium triacetoxyborohydride (3.2 g, 15 mmol) in THF 10 mL was stirred at ambient temperature for 12 h. The reaction mixture was diluted with EtOAc and washed with 5% aqueous NaHCO₃ (10 mL) and brine (10 mL), dried over Na₂SO₄ and concentrated in vacuo to provide pure 3-(((furan-2-ylmethyl)amino)methyl)-7-methoxy-quinolin-2(1H)-one as a white solid without purification in 90% yield.

A mixture of 3-(((furan-2-ylmethyl)amino)methyl)-7-methoxyquinolin-2(1H)-one (85 mg, 0.3 mmol) was dissolved in dichloromethane 6.0 mL followed by the addition of N,N-diisopropylethylamine (0.1 mL, 0.6 mmol) and benzoyl chloride (38 uL, 0.6 mmol). The reaction mixture was stirred for 3 h at 21° C. and concentrated in vacuo. The crude product was dissolved in DCM (0.5 mL) and purified by flash chromatography eluting with 50% DCM in EtOAc to afford the pure N-(furan-2-ylmethyl)-N-((7-methoxy-2-oxo-1,2-dihydroquinolin-3-yl)methyl)benzamide (JXC110) as a white solid in 70% yield.

### N-(Furan-2-ylmethyl)-N-((7-methoxy-2-oxo-1,2-dihydroquinolin-3-yl)methyl)benzamide (JXC010)

¹H NMR (500 MHz, DMSO-d₆) δ 11.71 (s, 1H), 7.59 (m, 4H), 7.47 (m, 2H), 7.38 (m, 2H), 6.79 (m, 2H), 6.32 (m, 2H), 4.61 (s, 2H), 4.31 (s, 2H), 3.78 (s, 3H).

¹³C NMR (125 MHz, CD₃OD) δ 171.6, 171.4, 162.4, 161.8, 161.3, 161.1, 151.1, 150.5, 143.6, 143.1, 140.1, 136.6, 135.7, 130.0, 129.8, 129.7, 128.9, 127.4, 126.7, 125.2, 125.0, 113.7, 111.3, 111.2, 111.0, 109.4, 109.1, 98.2, 55.8, 48.4, 46.7, 43.9, 41.5 (amide bond rotation causes most of the carbon peaks to be doubled).

### Example 2: Preparation of JHD Series

### General Procedure A: Preparation of 2-Oxo-1,2-dihydroquinoline-3-carbaldehydes

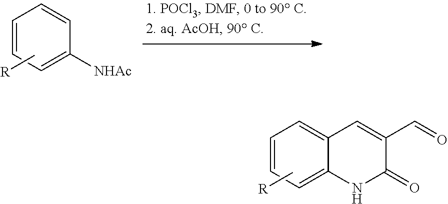

Phosphoryl chloride (7 eq.) was added dropwise to DMF (3 eq.) cooled at 0° C. The mixture was then stirred at 0° C. for 30 min. The N-phenylacetamide (1 eq.) was added in 4 portions over 15 min. The resultant yellow solution was warmed to 21° C. and stirred for 30 min. Next, it was stirred at 90° C. for 6 h, then cooled to 21° C. and poured onto ice. The mixture was extracted with chloroform (3×). The combined organic phases were washed with saturated NaHCO₃ solution, water and brine, dried over MgSO₄ and concentrated to give the crude 2-chloroquinoline-3-carbaldehyde. The aldehyde (˜1M) was stirred in aqueous AcOH (90%) overnight, cooled to 21° C. then at 0° C. for 1 h. The mixture was filtered. The solid was washed with saturated NaHCO₃ solution and water, then dried to constant weight to give the crude 2-oxo-1,2-dihydroquinoline-3-carbaldehyde.

### General Procedure B: Preparation of 3-(((Aryl/alkyl-methyl)amino)methyl)quinolin-2-yl benzoates

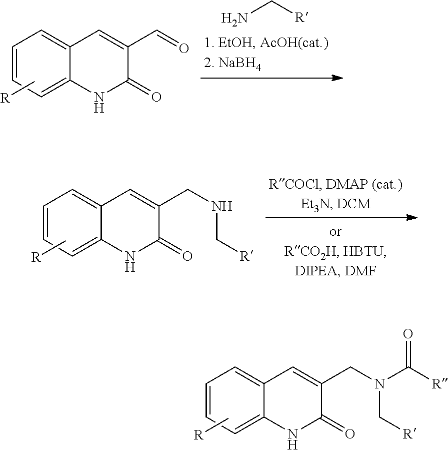

A suspension of 2-oxo-1,2-dihydroquinoline-3-carbaldehyde (1 eq.) and the aryl/alkyl-methylamine (1.4 eq.) in anhydrous EtOH (˜0.7 M, with a drop of AcOH) was stirred at 95° C. for 5 h, then cooled to 21° C. NaBH₄ (1.2 eq.) was added in one portion. The mixture was stirred at 21° C. for 15 h then quenched by saturated NaHCO₃ solution. The mixture was exacted with chloroform (3×) and washed with brine, dried over MgSO₄ and concentrated to give the crude amine, which was converted to the quinolinyl benzoate following one of the two procedures (B1 or B2). B1: To a solution of the crude amine (1 eq.) and Et₃N (3 eq.) in dichloromethane (about 0.1 M, with a crystal of DMAP) cooled at 0° C. was added the acid chloride (R″COCl, 1.2 eq). The mixture was warmed to 21° C. and stirred for 4 h. It was quenched with saturated NH₄Cl solution and exacted with chloroform (3×) to give the crude product, which was purified by flash chromatography (SiO₂, 1:1 to 1:2 hexanes-EtOAc) to give the desired product. B2: To a solution of the crude amine (1 eq.), HBTU (1 eq.) and DIPEA (1 eq.) in DMF (˜0.3 M) cooled at 0° C. was added the acid (R″CO₂H, 1 eq.). The mixture was warmed to 21° C. and stirred for 4 h. It was quenched with saturated NH₄Cl solution and exacted with EtOAc (3×) to give the crude product, which was purified by flash chromatography (SiO₂, 1:1 to 1:2 hexanes-EtOAc) to give the desired product.

The following compounds were prepared following General procedure B1: JHD009, JHD019 and JHD030.

The following compounds were prepared following General procedure B2: JHD034-JHD040, and JHD053.

### General Procedure C: Preparation of 5,6-Diaryl-1,2,4-triazine-3-thiol

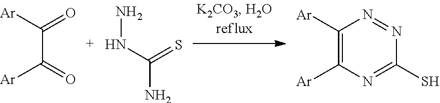

A mixture of the benzil compound (1 eq.), thiosemicarbazide (1 eq.) and K₂CO₃ (1.5 eq.) in water (0.1 M, calcd.) was refluxed overnight (˜21 h), then cooled to 21° C. AcOH was added until pH=5. The resultant solid was filtered and washed with water (4×), then dried to constant weight to give the desired 1,2,4-triazine-3-thiol.

### General Procedure D: Preparation of 2-((5,6-Diaryl-1,2,4-triazin-3-yl)thio)-1-phenylethan-1-ones

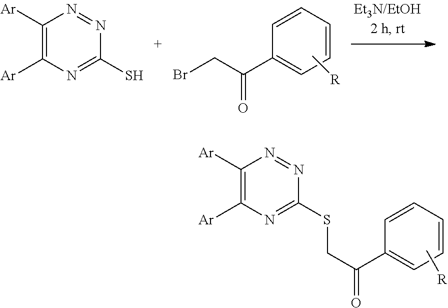

A suspension of the 1,2,4-triazine-3-thiol (1 eq.), the 2-bromoacetophenone (1 eq.) and Et₃N (2 eq.) was stirred in anhydrous EtOH (0.1 M, calcd.) at 21° C. for 2 h. It was diluted with DMF and partitioned between EtOAc and water. The organic phase was washed with water (4×) and brine, dried over MgSO₄, concentrated and purified by flash chromatography (SiO₂, 4:1 to 2:1 hexanes-EtOAc) to give the desired product.

The following compounds were prepared following General procedure D: JHD041-JHD044, JHD047, JHD048, JHD050, and JHD051.

### General Procedure E: Preparation of 2-((5,6-Diaryl-1,2,4-triazin-3-yl)thio)-N-phenylacet-amides

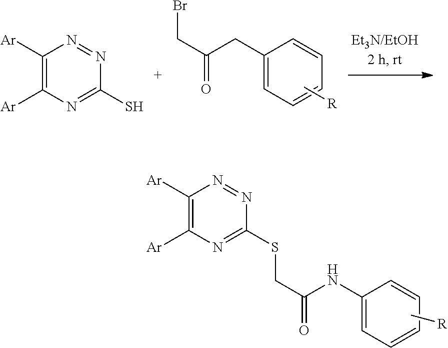

A suspension of the 1,2,4-triazine-3-thiol (1 eq.), the 2-bromo-N-arylacetamide (1 eq.) and Et₃N (2 eq.) was stirred in anhydrous EtOH (0.1 M, calcd.) at 21° C. for 2 h. It was diluted with DMF and partitioned between EtOAc and water. The organic phase was washed with water (4×) and brine, dried over MgSO₄, concentrated and purified by flash chromatography (SiO₂, 2:1 to 1:2 hexanes-EtOAc) to give the desired product.

The following compound were prepared following General procedure E: JHD053.

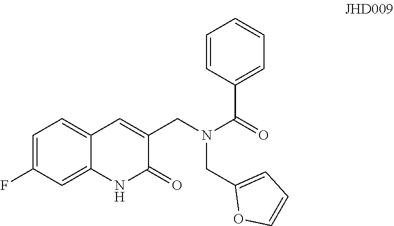 [SMILES: O=C(c1ccccc1)N(Cc1ccco1)Cc1cc2ccc(F)cc2[nH]c1=O]

Following General procedure B, 184 mg was obtained (26% overall yield). ¹H NMR (500 MHz, CDCl₃; mixture of rotamers): δ (ppm) 12.10 (br s, 0.42H), 12.01 (br s, 0.58H), 7.98 (br s, 1H), 7.72-7.34 (m, 7H), 7.16-6.90 (m, 2H), 6.52-6.21 (m, 2H), 4.97-4.48 (m, 4H); ¹³C NMR (125 MHz, CDCl₃): δ (ppm) 172.1, 164.8, 150.5, 150.2, 142.7, 142.4, 139.1, 139.0, 135.95, 135.89, 130.2, 130.1, 129.8, 127.46, 127.38, 127.2, 126.6, 116.7, 111.5, 110.4, 101.7, 101.5, 47.25, 43.69, 29.7, 22.7, 14.1; LRMS (ESI) m/z: [M+H]⁺ Calcd for C₂₂H₁₈FN₂O₃: 377.12; Found 377.17.

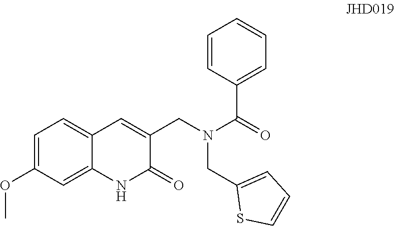 [SMILES: COc1ccc2cc(CN(Cc3cccs3)C(=O)c3ccccc3)c(=O)[nH]c2c1]

Following General procedure A, 8 mg was obtained (10% overall yield). ¹H NMR (500 MHz, CDCl₃; mixture of rotamers): δ (ppm) 11.72 (br s, 0.43H), 11.52 (br s, 0.57H), 7.9-7.71 (m, 2H), 7.57-7.42 (m, 6H), 7.08-6.67 (m, 4H), 4.96 (br s, 1H), 4.87 (br s, 1H), 4.69 (br s, 1H), 4.48 (br s, 1H), 3.84 (s, 3H); ¹³C NMR (125 MHz, CDCl₃): δ (ppm) 169.5, 161.9, 140.8, 139.6, 139.5, 133.3, 132.1, 129.9, 129.1, 128.6, 128.5, 127.4, 126.9, 125.9, 114.1, 112.7, 112.6, 98.1, 55.9, 55.7, 43.6, 31.9, 29.7, 29.4, 22.7, 14.2; LRMS (ESI) m/z: [M+H]⁺ Calcd for C₂₃H₂₁N₂O₃S: 405.12; Found 405.17.

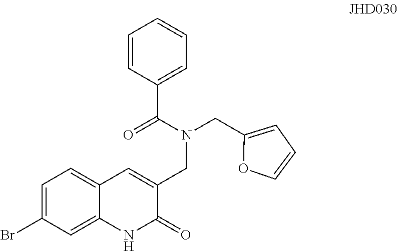 [SMILES: O=C(c1ccccc1)N(Cc1ccco1)Cc1cc2ccc(Br)cc2[nH]c1=O]

Following General procedure A, 3 mg was obtained (4% overall yield). ¹H NMR (500 MHz, CDCl₃; mixture of rotamers): δ (ppm) 7.99 (br s, 0.73H), 7.83 (br s, 0.27H), 7.56-7.30 (m, 8H), 7.05-6.97 (m, 1H), 6.34-6.03 (m, 2H), 4.80-4.35 (m, 4H); LRMS (ESI) m/z: [M+H]⁺ Calcd for C₂₂H₁₈BrN₂O₃: 437.04; Found 437.14.

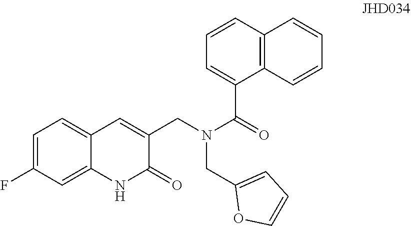 [SMILES: O=C(c1cccc2ccccc12)N(Cc1ccco1)Cc1cc2ccc(F)cc2[nH]c1=O]

Following General procedure B, 3 mg was obtained (6% overall yield). ¹H NMR (500 MHz, CD₃OD; mixture of rotamers): δ (ppm) 8.30-6.51 (m, 13H), 6.46-6.54 (m, 2H), 4.78-3.44 (m, 4H); ¹³C NMR (125 MHz, CD₃OD; DEPT-135): δ (ppm) 137.9, 136.9, 129.5, 128.9, 128.3, 128.1, 127.9, 127.4, 126.5, 124.1, 114.3, 110.4, 109.3, 109.0, 108.5, 100.2, 47.7, 47.6, 47.2, 46.6, 46.5, 46.2, 43.5, 43.4, 38.7; LRMS (ESI) m/z: [M+H]⁺ Calcd for C₂₆H₂₀FN₂O₃: 427.14; Found 427.21.

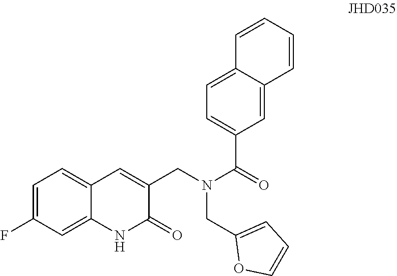 [SMILES: O=C(c1ccc2ccccc2c1)N(Cc1ccco1)Cc1cc2ccc(F)cc2[nH]c1=O]

Following General procedure B, 4 mg was obtained (9% overall yield). ¹H NMR (500 MHz, CD₃OD; mixture of rotamers): δ (ppm) 8.50-7.32 (m, 5H), 7.30-6.82 (m, 4H), 6.45-6.14 (m, 2H), 4.90 (br s, 1H), 4.80-4.34 (m, 3H); ¹³C NMR (125 MHz, CD₃OD; DEPT-135): δ (ppm) 142.1, 136.5, 136.3, 129.5, 127.8, 126.7, 124.5, 122.8, 110.2, 109.4, 108.9, 100.4, 48.0, 46.7, 43.5, 41.2, 38.3; LRMS (ESI) m/z: [M+H]⁺ Calcd for C₂₆H₂₀FN₂O₃: 427.14; Found 427.21.

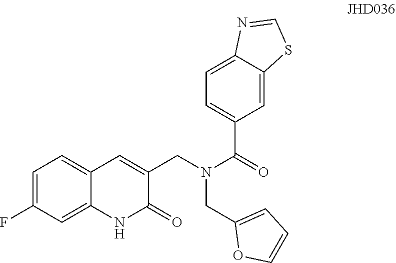 [SMILES: O=C(c1ccc2ncsc2c1)N(Cc1ccco1)Cc1cc2ccc(F)cc2[nH]c1=O]

Following General procedure B, 3 mg was obtained (6% overall yield). ¹H NMR (500 MHz, CD₃OD; mixture of rotamers): δ (ppm) 9.50-9.18 (m, 1H), 8.44-7.49 (m, 5H), 7.42 (br s, 1H), 7.24-6.83 (m, 2H), 6.54-6.12 (m, 2H), 4.90 (br s, 1H), 4.66 (br s, 2H), 4.47 (br s, 1H); ¹³C NMR (125 MHz, CD₃OD; DEPT-135): δ (ppm) 141.5, 136.9, 136.6, 129.7, 124.8, 123.8, 122.0, 121.0, 120.5, 110.3, 109.8, 109.0, 100.4, 48.5, 47.0, 46.4, 43.9, 41.2; LRMS (ESI) m/z: [M+H]⁺ Calcd for C₂₃H¹⁷FN₃O₃S: 434.09; Found 434.14.

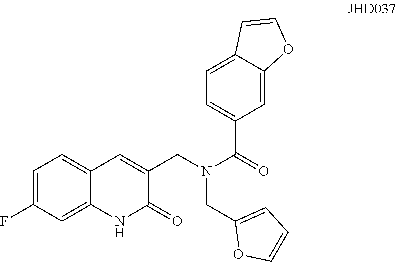 [SMILES: O=C(c1ccc2ccoc2c1)N(Cc1ccco1)Cc1cc2ccc(F)cc2[nH]c1=O]

Following General procedure B, 4 mg was obtained (9% overall yield). ¹H NMR (500 MHz, CDCl₃, mixture of rotamers): δ (ppm) 11.31-10.78 (m, 1H), 8.22-7.31 (m, 7H), 7.12-6.63 (m, 3H), 6.48-6.11 (m, 2H), 4.96-4.55 (m, 4H); ¹³C NMR (125 MHz, CDCl₃; DEPT-135): δ (ppm) 147.5, 145.6, 141.8, 139.0, 129.8, 123.9, 121.7, 121.5, 120.9, 113.2, 111.3, 110.6, 110.2, 109.4, 108.7, 106.4, 106.1, 101.3, 48.0, 46.8, 44.4, 43.3; LRMS (ESI) m/z: [M+H]⁺ Calcd for C₂₄H₁₈FN₂O₄: 417.12; Found 417.17.

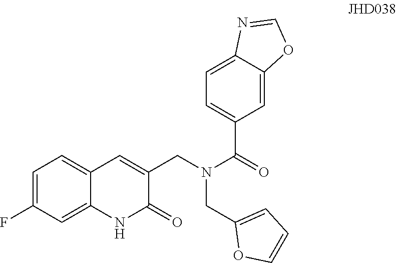 [SMILES: O=C(c1ccc2ncoc2c1)N(Cc1ccco1)Cc1cc2ccc(F)cc2[nH]c1=O]

Following General procedure B, 3 mg was obtained (7% overall yield). ¹H NMR (500 MHz, CDCl₃; mixture of rotamers): δ (ppm) 11.29 (br s, 1H), 8.38-7.29 (m, 7H), 7.12-6.76 (m, 2H), 6.33 (br s, 2H), 5.17-4.32 (m, 4H); ¹³C NMR (125 MHz, CDCl₃; DEPT-135): δ (ppm) 151.8, 145.3, 141.0, 139.4, 130.0, 123.4, 122.7, 120.6, 111.7, 109.8, 109.5, 101.1, 48.2, 47.2, 43.5, 42.3; LRMS (ESI) m/z: [M+H]⁺ Calcd for C₂₃H₁₇FN₃O₄: 418.11; Found 418.16.

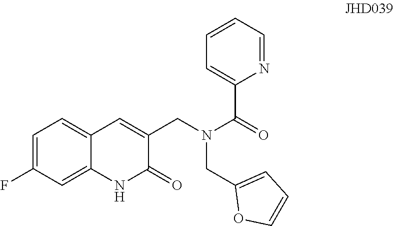 [SMILES: O=C(c1ccccn1)N(Cc1ccco1)Cc1cc2ccc(F)cc2[nH]c1=O]

Following General procedure B, 2 mg was obtained (5% overall yield). ¹H NMR (500 MHz, CDCl₃; mixture of rotamers): δ (ppm) 11.26-10.63 (m, 1H), 8.83-8.40 (m, 1H), 8.21-7.30 (m, 6H), 7.16-6.77 (m, 2H), 6.59-5.89 (m, 2H), 5033-4.47 (m, 4H); ¹³C NMR (125 MHz, CDCl₃; DEPT-135): δ (ppm) 147.7, 145.1, 140.7, 137.8, 136.4, 129.7, 125.8, 124.5, 123.3, 111.7, 110.6, 109.4, 108.8, 108.4, 101.3, 47.1, 46.4, 43.1, 42.0, 38.8; LRMS (ESI) m/z: [M+H]⁺ Calcd for C₂₁H₁₇FN₃O₃: 378.12; Found 378.17.

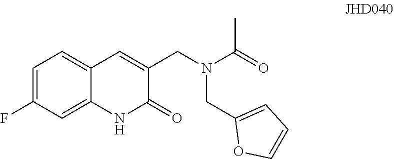 [SMILES: CC(=O)N(Cc1ccco1)Cc1cc2ccc(F)cc2[nH]c1=O]

Following General procedure A, 4 mg was obtained (9% overall yield). ¹H NMR (500 MHz, CDCl₃; mixture of rotamers): δ (ppm) 8.18-7.74 (m, 1H), 7.71-7.29 (m, 3H), 7.16-6.77 (m, 2H), 6.46-6.08 (m, 2H), 5.16-4.20 (m, 4H); ¹³C NMR (125 MHz, CDCl₃): δ (ppm) 170.9, 169.3, 150.3, 149.5, 142.9, 142.7, 142.5, 140.3, 138.6, 136.0, 130.5, 129.4, 124.6, 123.0, 117.5, 117.2, 112.2, 111.3, 110.5, 110.4, 109.5, 108.2, 101.5, 101.2, 47.1, 43.9, 42.6, 41.9, 37.9, 21.8, 21.2, 20.4; LRMS (ESI) m/z: [M+H]⁺ Calcd for C₁₇H₁₆FN₂O₃: 315.11; Found 315.16.

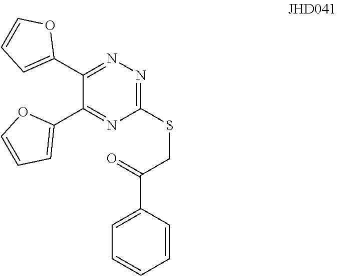 [SMILES: O=C(CSc1nnc(-c2ccco2)c(-c2ccco2)n1)c1ccccc1]

Following General procedure D, 23 mg was obtained (32% yield). ¹H NMR (500 MHz, CDCl₃): δ (ppm) 8.17-8.07 (m, 2H), 7.64-7.60 (m, 1H), 7.59 (dd, J=1.7, 0.7 Hz, 1H), 7.55 (dd, J=1.7, 0.7 Hz, 1H), 7.53-7.46 (m, 2H), 6.99 (dd, J=3.4, 0.8 Hz, 1H), 6.75 (dd, J=3.6, 0.7 Hz, 1H), 6.61 (dd, J=3.4, 1.8 Hz, 1H), 6.50 (dd, J=3.6, 1.7 Hz, 1H), 4.85 (s, 2H); ¹³C NMR (125 MHz, CDCl₃): δ (ppm) 192.9, 169.4, 148.1, 148.0, 147.0, 144.11, 144.10, 142.9, 135.9, 133.7, 128.8, 128.6, 118.6, 112.85, 112.77, 112.1, 36.6; LRMS (ESI) m/z: [M+H]⁺ Calcd for C₁₉H₁₄N₃O₃S: 364.07; Found 364.18.

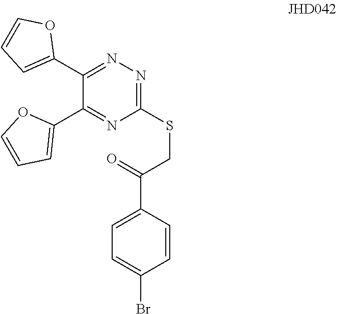 [SMILES: O=C(CSc1nnc(-c2ccco2)c(-c2ccco2)n1)c1ccc(Br)cc1]

Following General procedure D, 49 mg was obtained (55% yield). ¹H NMR (500 MHz, CDCl₃): δ (ppm) 7.98-7.91 (m, 2H), 7.70-7.62 (m, 2H), 7.59 (dd, J=1.8, 0.8 Hz, 1H), 7.56 (dd, J=1.7, 0.7 Hz, 1H), 6.99 (dd, J=3.4, 0.7 Hz, 1H), 6.75 (dd, J=3.6, 0.7 Hz, 1H), 6.61 (dd, J=3.4, 1.8 Hz, 1H), 6.51 (dd, J=3.6, 1.7 Hz, 1H), 4.79 (s, 2H); ¹³C NMR (125 MHz, CDCl₃): δ (ppm) 192.1, 169.1, 148.0, 147.9, 147.1, 144.16, 144.14, 143.0, 134.7, 132.1, 130.1, 128.9, 118.7, 112.90, 112.85, 112.2, 38.3; LRMS (ESI) m/z: [M+H]⁺ Calcd for C₁₉H₁₃BrN₃O₃S: 441.98; Found 442.10.

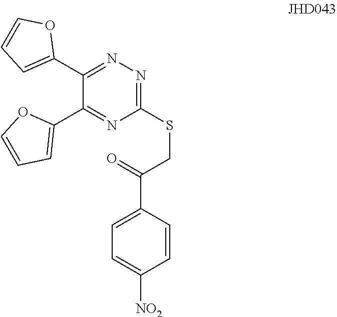 [SMILES: O=C(CSc1nnc(-c2ccco2)c(-c2ccco2)n1)c1ccc([N+](=O)[O-])cc1]

Following General procedure D, 28 mg was obtained (34% yield). ¹H NMR (500 MHz, CDCl₃): δ (ppm) 8.38-8.31 (m, 2H), 8.28-8.20 (m, 2H), 7.59 (dd, J=0.8, 0.8 Hz, 1H), 7.55 (dd, J=0.8, 0.8 Hz, 1H), 6.99 (d, J=3.5 Hz, 1H), 6.75 (d, J=3.6 Hz, 1H), 6.62 (dd, J=3.5, 1.8 Hz, 1H), 6.53 (dd, J=3.6, 1.7 Hz, 1H), 4.82 (s, 2H); ¹³C NMR (125 MHz, CDCl₃): δ (ppm) 191.9, 168.7, 150.6, 147.83, 147.76, 147.3, 144.3, 143.2, 140.6, 129.6, 124.0, 118.9, 113.01, 112.99, 112.2, 38.5; LRMS (ESI) m/z: [M+H]⁺ Calcd for C₁₉H₁₃N₄O₅S: 409.05; Found 409.15.

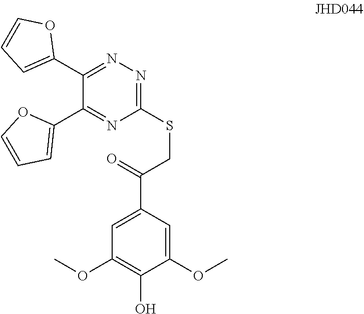 [SMILES: COc1cc(C(=O)CSc2nnc(-c3ccco3)c(-c3ccco3)n2)cc(OC)c1O]

Following General procedure D, 94 mg was obtained (50% yield). ¹H NMR (500 MHz, CDCl₃): δ (ppm) 7.61-7.53 (m, 2H), 7.38 (s, 2H), 6.99 (d, J=3.6 Hz, 1H), 6.78 (d, J=3.6 Hz, 1H), 6.61 (dd, J=3.4, 1.7 Hz, 1H), 6.52 (dd, J=3.6, 1.7 Hz, 1H), 6.00 (s, 1H), 4.85 (s, 2H), 3.95 (s, 6H); ¹³C NMR (125 MHz, CDCl₃): δ (ppm) 191.5, 169.5, 148.1, 148.0, 147.1, 146.9, 144.2, 144.1, 142.9, 140.3, 127.4, 118.6, 112.9, 112.8, 112.1, 106.0, 56.7, 38.2; LRMS (ESI) m/z: [M+H]⁺ Calcd for C₂₁H₁₈N₃O₆S: 440.08; Found 440.18.

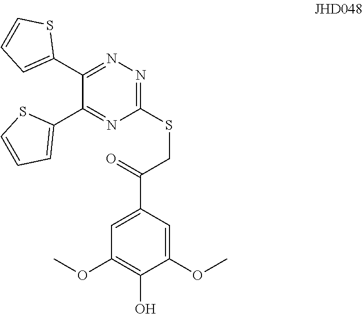 [SMILES: COc1cc(C(=O)CSc2nnc(-c3cccs3)c(-c3cccs3)n2)cc(OC)c1O]

Following General procedure D, 20 mg was obtained (59% yield). ¹H NMR (500 MHz, CDCl₃): δ (ppm) 7.45 (ddd, J=6.2, 5.0, 1.2 Hz, 2H), 7.40 (dd, J=3.9, 1.1 Hz, 1H), 7.38 (s, 2H), 7.37 (dd, J=3.6, 1.1 Hz, 1H), 7.11 (dd, J=5.0, 3.6 Hz, 1H), 6.97 (dd, J=5.0, 3.9 Hz, 1H), 6.01 (s, 1H), 4.84 (s, 2H), 3.96 (s, 6H); ¹³C NMR (125 MHz, CDCl₃): δ (ppm) 191.3, 169.1, 149.1, 147.1, 146.9, 140.3, 138.6, 136.5, 133.1, 132.4, 129.2, 129.1, 128.4, 127.7, 127.3, 106.0, 56.6, 38.3; LRMS (ESI) m/z: [M+H]⁺ Calcd for C₂₁H₁₈N₃O₄S₃: 472.04; Found 472.14.

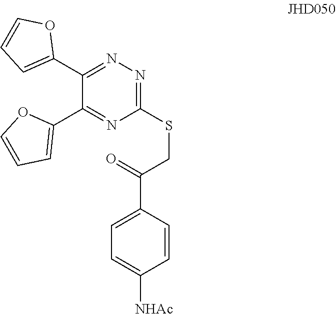 [SMILES: CC(=O)Nc1ccc(C(=O)CSc2nnc(-c3ccco3)c(-c3ccco3)n2)cc1]

Following General procedure D, 10 mg was obtained (15% yield). ¹H NMR (500 MHz, CDCl₃): δ (ppm) 8.01 (s, 1H), 7.99 (s, 1H), 7.65 (s, 1H), 7.63 (s, 1H), 6.94 (d, J=3.4 Hz, 1H), 6.72 (d, J=3.5 Hz, 1H), 6.58 (dd, J=3.5, 1.7 Hz, 1H), 6.47 (dd, J=3.5, 1.7 Hz, 1H), 4.77 (s, 2H), 2.21 (s, 1H), 2.15 (3, 3H); ¹³C NMR (125 MHz, CDCl₃): δ (ppm) 191.9, 169.5, 147.9, 147.2, 144.2, 144.1, 143.3, 142.8, 131.1, 129.9, 118.9, 118.8, 112.9, 112.8, 112.1, 38.3, 24.3; LRMS (ESI) m/z: [M+H]⁺ Calcd for C₂₁H₁₇N₄O₄S: 421.09; Found 421.18.

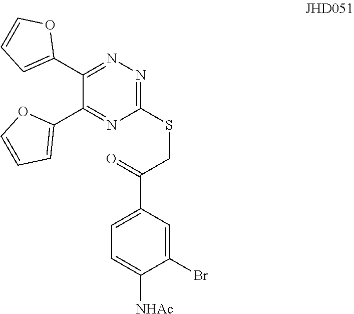 [SMILES: CC(=O)Nc1ccc(C(=O)CSc2nnc(-c3ccco3)c(-c3ccco3)n2)cc1Br]

Following General procedure D, 9 mg was obtained (11% yield). ¹H NMR (500 MHz, CDCl₃): δ (ppm) 8.57 (d, J=8.5 Hz, 1H), 8.27 (d, J=2.0 Hz, 1H), 8.05 (dd, J=8.5, 1.8 Hz, 1H), 7.84 (s, 1H), 7.61-7.56 (m, 2H), 6.99 (dd, 1=3.4, 0.7 Hz, 1H), 6.76 (dd, 1=3.5, 0.6 Hz, 1H), 6.61 (dd, J=3.4, 1.8 Hz, 1H), 6.52 (dd, J=3.6, 1.7 Hz, 1H), 4.78 (s, 2H), 2.29 (s, 3H); ¹³C NMR (125 MHz, CDCl₃): δ (ppm) 190.6, 169.1, 168.4, 148.0, 147.9, 147.2, 144.1, 143.0, 140.1, 132.7, 132.2, 129.8, 129.2, 120.4, 118.7, 112.9, 112.8, 112.1, 38.2, 25.2; LRMS (ESI) m/z: [M+H]⁺ Calcd for C₂₁H₁₇N₄O₄S: 499.00; Found 499.05.

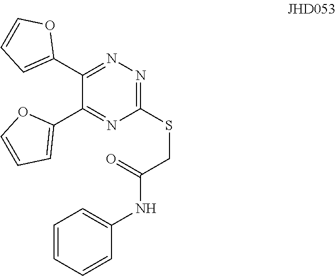 [SMILES: O=C(CSc1nnc(-c2ccco2)c(-c2ccco2)n1)Nc1ccccc1]

Following General procedure E, 19 mg was obtained (42% yield). ¹H NMR (500 MHz, CDCl₃): δ (ppm) 9.20 (s, 1H), 7.68 (dd, J=1.7, 0.7 Hz, 1H), 7.64 (dd, J=1.8, 0.8 Hz, 1H), 7.48-7.45 (m, 2H), 7.30-7.23 (m, 2H), 7.12-7.02 (m, 2H), 6.90 (dd, J=3.9, 0.5 Hz, 1H), 6.66 (dd, J=3.5, 1.8 Hz, 1H), 6.59 (dd, J=3.9, 1.7 Hz, 1H), 4.03 (s, 2H); ¹³C NMR (125 MHz, CDCl₃): δ (ppm) 169.7, 166.7, 147.66, 147.61, 147.59, 144.4, 144.2, 143.3, 137.8, 128.9, 124.4, 119.9, 119.7, 113.4, 113.3, 112.4, 35.3; LRMS (ESI) m/z: [M+H]⁺ Calcd for C₁₉H₁₅N₄O₃S: 379.08; Found 379.18.

### Preparation of JHD047

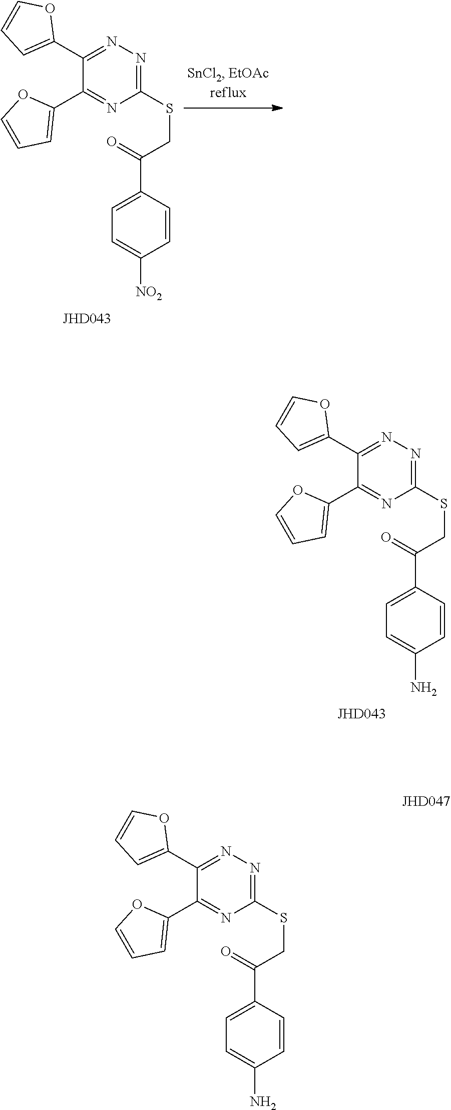 [SMILES: O=C(CSc1nnc(-c2ccco2)c(-c2ccco2)n1)c1ccc([N+](=O)[O-])cc1 ; Nc1ccc(C(=O)CSc2nnc(-c3ccco3)c(-c3ccco3)n2)cc1 ; Nc1ccc(C(=O)CSc2nnc(-c3ccco3)c(-c3ccco3)n2)cc1]

A solution of JHD043 (12 mg, 0.029 mmol) and tin (II) chloride monohydrate (33 mg, 0.15 mmol) in EtOAc (1.5 mL) was refluxed for 3 h. The dark mixture was cooled to 21° C., partitioned between water and EtOAc. The organic phase was washed with saturated NaHCO₃ solution, water and brine, then dried over MgSO₄ and concentrated. The crude was purified by PTLC (SiO₂, 1:2 Hex.-EtOAc) to give the desired JHD047 (1.6 mg, 15% yield). ¹H NMR (500 MHz, CDCl₃): δ (ppm) 7.95 (s, 1H), 7.93 (s, 1H), 7.59 (br s, 2H), 6.99 (d, J=3.0 Hz, 1H), 6.79 (d, J=3.5 Hz, 1H), 6.68 (s, 1H), 6.66 (s, 1H), 6.62-6.59 (m, 1H), 6.52-6.49 (m, 1H), 4.80 (s, 2H), 4.17 (br s, 2H); ¹³C NMR (125 MHz, CDCl₃; DEPT-135): δ (ppm) 130.9, 117.8, 113.3, 112.8, 112.1, 111.9, 38.5; LRMS (ESI) m/z: [M+H]⁺ Calcd for C₁₉H₁₄N₄O₃S: 379.05; Found 379.15.

### Preparation of JHD045 and JH046

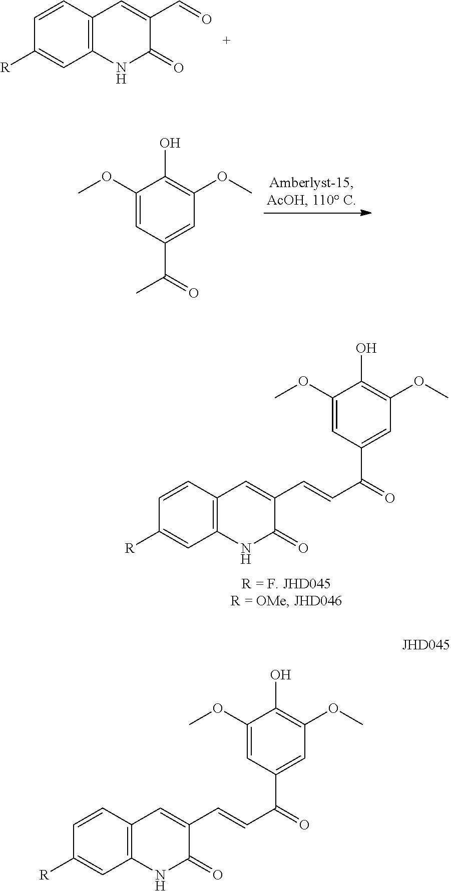

A mixture of the aldehyde (0.3 mmol), acetosyringone (0.2 mmol) and Amberlyst-15 (250 mg) in AcOH (2.5 mL) was stirred at 110° C. for 30 min. The mixture was concentrated and the residue was purified by flash chromatography (SiO₂, 50:1 CH₂Cl₂-MeOH) to give the desired product.

5 mg, 5% yield. ¹H NMR (500 MHz, CDCl₃): δ (ppm) 9.76 (br s, 1H), 8.47 (d, J=15.1 Hz, 1H), 8.01 (s, 1H), 7.71 (d, J=15.1 Hz, 1H), 7.68-7.57 (m, 1H), 7.41 (s, 2H), 7.08-6.87 (m, 2H), 4.00 (s, 6H); ¹³C NMR (125 MHz, CDCl₃; DEPT-135): δ (ppm) 141.9, 138.1, 135.0, 130.8, 125.4, 110.9, 107.7, 105.5, 101.1, 56.3; LRMS (ESI) m/z: [M+H]⁺ Calcd for C₂₀H₁₇FNO₅: 370.10; Found 370.15.

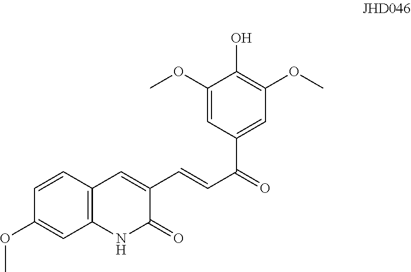 [SMILES: COc1ccc2cc(/C=C/C(=O)c3cc(OC)c(O)c(OC)c3)c(=O)[nH]c2c1]

10 mg, 14% yield. ¹H NMR (500 MHz, DMSO-d₆): δ (ppm) 11.89 (br s, 1H), 9.43 (s, 1H), 8.51 (s, 1H), 8.17 (d, J=15.4 Hz, 1H), 7.78 (d, J=15.4 Hz, 1H), 7.63 (d, J=7.5 Hz, 1H), 7.35 (s, 2H), 7.02-6.58 (m, 2H), 4.12-3.68 (m, 9H); ¹³C NMR (125 MHz, DMSO-d₆; DEPT-135): δ (ppm) 140.5, 138.3, 130.6, 122.3, 111.6, 106.6, 97.4, 56.2, 55.4; LRMS (ESI) m/z: [M+H]⁺ Calcd for C₂₁H₂₀NO₆: 382.12; Found 382.17.

### Example 3: Preparation of JYX Series

General: THF was distilled from sodium under an argon atmosphere. Methylene chloride was distilled from calcium hydride under an argon atmosphere. All other solvents or reagents were purified according to literature procedures. ¹H NMR spectra were recorded on Bruker spectrometers at 400 MHz or 500 MHz and are reported relative to deuterated solvent signals. Data for 1H NMR spectra are reported as follows: chemical shift (δ ppm), multiplicity, coupling constant (Hz) and integration. Splitting patterns are designated as follows: s, singlet; d, doublet; t, triplet; q, quartet; m, multiplet; and br, broad. ¹³C NMR spectra were recorded on Bruker Spectrometers at 100 MHz or 125 MHz. Data for ¹³C NMR spectra are reported as follows: chemical shift (δ ppm), multiplicity and coupling constant (Hz). Splitting patterns are designated as the same in 1H NMR. High resolution mass spectrometry was taken on a Thermo Fisher Scientific Exactive Plus mass spectrometer equipped with an IonSense ID-CUBE DART ion source.

### General Procedure A Exemplified by the Synthesis of 2-Amino-3-hydroxy-N-(2,3,4-trihydroxyphenethyl)propanamide, JYX001

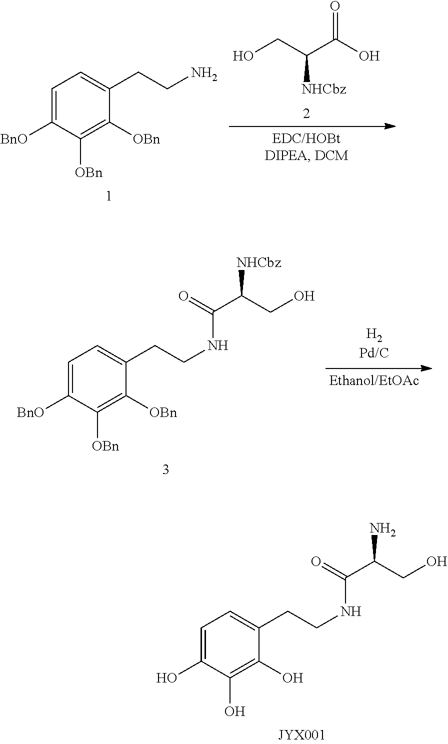 [SMILES: NCCc1ccc(OCc2ccccc2)c(OCc2ccccc2)c1OCc1ccccc1 ; O=C(N[C@@H](CO)C(=O)O)OCc1ccccc1 ; O=C(N[C@@H](CO)C(=O)NCCc1ccc(OCc2ccccc2)c(OCc2ccccc2)c1OCc1ccccc1)OCc1ccccc1 ; N[C@@H](CO)C(=O)NCCc1ccc(O)c(O)c1O]

To a solution of 1 (0.75 g, 1.7 mmol), 2 (0.61 g, 2.55 mmol) and 1-hydroxybenzotriazole hydrate (HOBT, 0.39 g, 2.55 mmol) in anhydrous dichloromethane (15.0 ml) was added diisopropyl-ethylamine (DIPEA, 1.5 ml) at 0° C. under argon. After stirring at 0° C. for 15 min, a suspension of EDC·HCl (0.49 g, 2.55 mmol) in dichloromethane (5.0 ml) was added dropwise. Then the mixture was warmed to 21° C. and stirred for 16 h. The mixture was diluted with dichloro-methane, washed with saturated sodium bicarbonate, dried over anhydrous sodium sulfate, and concentrated under reduced pressure. The resulting residue was purified by flash choreography on silica gel (dichloromethane/MeOH) to give compound 3 (0.71 g, 63%). Compound 3 (0.71 g, 1.07 mmol) was dissolved in a mixture of ethanol (10.0 ml) and ethyl acetate (10.0 ml) and 5%-Pd/C (115 mg, 0.054 mmol) was then added. The resulting mixture was then stirred at 21° C. for 12 h under hydrogen. The reaction mixture was filtered through a Celite plug, and the solvent was removed under reduced pressure. The resulting residue was purified by flash chromatography on silica gel (dichloromethane/MeOH) to give JYX001 (156 mg, 57%). ¹H NMR (400 MHz, CD₃OD) δ 6.43 (d, J=8.4 Hz, 1H), 6.27 (d, J=8.4 Hz, 1H), 3.73 (dd, J=11.2, 4.8 Hz, 1H), 3.62 (dd, J=10.8, 6.4 Hz, 1H), 3.51 (dd, J=6.4, 4.8 Hz, 1H), 3.42-3.37 (m, 2H), 2.73 (t, J=7.0 Hz, 2H); ¹³C NMR (100 MHz, CD₃OD) δ 175.1, 145.8, 145.5, 134.3, 121.4, 118.7, 107.9, 65.4, 57.8, 41.4, 30.7; FIRMS (ESI, m/z): calcd for([M+H]+). Found:.

### Synthesis and Characterization of (S)-2-amino-N-(2,4-dihydroxyphenethyl)-3-hydroxypropanamide, JYX002

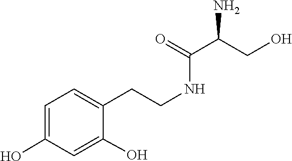 [SMILES: N[C@@H](CO)C(=O)NCCc1ccc(O)cc1O]

This product was prepared according to general procedure A. ¹H NMR (400 MHz, CD₃OD) δ 6.84 (d, J=8.0 Hz, 1H), 6.26 (d, J=2.4 Hz, 1H), 6.20 (dd, J=8.0, 2.4 Hz, 1H), 3.73 (dd, J=11.2, 4.4 Hz, 1H), 3.62 (dd, J=11.2, 6.4 Hz, 1H), 3.53 (dd, J=6.4, 4.8 Hz, 1H), 3.42-3.33 (m, 2H), 2.69 (t, J=7.2 Hz, 2H); ¹³C NMR (100 MHz, CD₃OD) δ 172.3, 158.1, 157.4, 132.1, 117.7, 107.5, 103.5, 64.0, 57.2, 41.1, 30.4; FIRMS (ESI, m/z): calcd for C₁₁H₁₅N₂O₄ ([M−H]⁻): 239.1037, Found: 239.1029.

### Synthesis and Characterization of (S)-2-Amino-N-(2,4-difluoro-3-hydroxyphenethyl)-3-hydroxypropanamide, JYX003

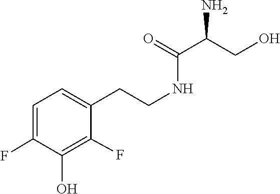 [SMILES: N[C@@H](CO)C(=O)NCCc1ccc(F)c(O)c1F]

This product was prepared according to general procedure A. ¹H NMR (400 MHz, d₆-DMSO) δ 7.97 (t, J=5.6 Hz, 1H), 6.87 (td, J=9.6, 1.6 Hz, 1H), 6.64-6.58 (m, 1H), 3.45 (dd, J=10.4, 4.8 Hz, 1H), 3.32 (dd, J=10.4, 6.4 Hz, 1H), 3.25-3.19 (m, 2H), 3.16 (dd, J=6.4, 4.4 Hz, 1H), 2.64 (t, J=7.0 Hz, 2H); ¹³C NMR (100 MHz, d₆-DMSO) δ 173.0, 151.5 (dd, J=239.0, 12.7 Hz), 151.4 (dd, J=239.4, 13.4 Hz), 134.2 (t, J=16.5 Hz), 123.0 (dd, J=14.2, 3.1 Hz), 119.3 (dd, J=7.6, 6.2 Hz), 111.4 (dd, J=18.0, 3.4 Hz), 64.4, 57.2, 39.0, 28.9; HRMS (ESI, m/z): calcd for C₁₁H₁₃F₂N₂O₃ ([M−H]⁻): 259.0900, Found: 259.0896.

### Synthesis and Characterization of (S)-2-Amino-N-(3-fluoro-2,4-dihydroxyphenethyl)-3-hydroxypropanamide, JYX004

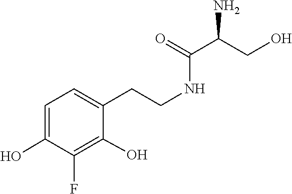 [SMILES: N[C@@H](CO)C(=O)NCCc1ccc(O)c(F)c1O]

This product was prepared according to general procedure A. ¹H NMR (400 MHz, CD₃OD) δ 6.65 (dd, J=8.4, 2.0 Hz, 1H), 6.30 (t, J=8.2 Hz, 1H), 3.65 (dd, J=10.8, 4.8 Hz, 1H), 3.56 (dd, J=10.8, 6.0 Hz, 1H), 3.39-3.33 (m, 3H), 2.72 (t, J=7.2 Hz, 2H); ¹³C NMR (100 MHz, CD₃OD) δ 175.0, 145.4 (d, J=11.1 Hz), 145.0 (d, J=11.9 Hz), 143.0 (d, J=230.9 Hz), 125.5 (d, J=3.8 Hz), 120.0, 108.7 (d, J=1.2 Hz), 65.4, 57.8, 40.8, 30.5 (d, J=2.3 Hz); HRMS (ESI, m/z): calcd for C₁₁H₁₆FN₂O₄ ([M+H]⁺): 259.1089, Found: 259.1075.

### Synthesis and Characterization of (S)-2-Amino-N-(2,4-dihydroxy-3-methoxyphenethyl)-3-hydroxypropanamide, JYX005

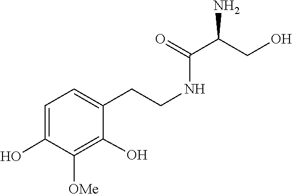 [SMILES: COc1c(O)ccc(CCNC(=O)[C@@H](N)CO)c1O]

This product was prepared according to general procedure A. ¹H NMR (400 MHz, CD₃OD) δ 6.63 (d, J=8.4 Hz, 1H), 6.26 (d, J=8.4 Hz, 1H), 3.75 (s, 3H), 3.66 (dd, J=10.8, 4.8 Hz, 1H), 3.57 (dd, J=11.2, 6.4 Hz, 1H), 3.40-3.35 (m, 3H), 2.71 (t, J=7.2 Hz, 2H); ¹³C NMR (100 MHz, CD₃OD) δ 174.5, 150.2, 149.8, 136.9, 126.1, 118.6, 108.1, 65.1, 60.8, 57.7, 41.0, 30.6; HRMS (ESI, m/z): calcd for C₁₂H₁₇N₂O₅ ([M+H]⁺): 269.1143, Found: 269.1124.

### Synthesis and Characterization of (S)-2-Amino-3-hydroxy-N-(2,3,4-tris(2-hydroxy-ethoxy)phenethyl)propanamide, JYX006

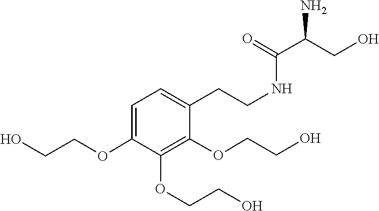 [SMILES: N[C@@H](CO)C(=O)NCCc1ccc(OCCO)c(OCCO)c1OCCO]

This product was prepared according to general procedure A. 1H NMR (400 MHz, CD₃OD) δ 6.88 (d, J=8.4 Hz, 1H), 6.72 (d, J=8.4 Hz, 1H), 4.12-4.08 (m, 4H), 4.04-4.01 (m, 2H), 3.86-3.82 (m, 4H), 3.76-3.74 (m, 2H), 3.63 (dd, J=10.8, 4.8 Hz, 1H), 3.56 (dd, J=10.8, 6.0 Hz, 1H), 3.38 (t, J=7.2 Hz, 2H), 3.32-3.30 (m, 1H), 2.79 (t, J=7.0 Hz, 2H); ¹³C NMR (100 MHz, CD₃OD) δ 175.6, 153.2, 152.5, 142.8, 127.0, 126.1, 110.7, 76.2, 76.0, 71.9, 85.6, 62.6, 62.3, 61.7, 58.0, 41.5, 30.6; HRMS (ESI, m/z): calcd for C₁₇H₂₉N₂O₈ ([M+H]⁺): 389.1918, Found: 389.1907.

### Synthesis and Characterization of (S)-2-Amino-3-hydroxy-N-(3,4,5-trihydroxyphenethyl)propanamide, JYX007

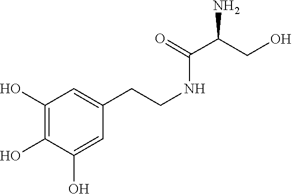 [SMILES: N[C@@H](CO)C(=O)NCCc1cc(O)c(O)c(O)c1]

This product was prepared according to general procedure A. 1H NMR (400 MHz, CD₃OD) δ 6.19 (s, 2H), 3.64 (dd, J=10.8, 4.8 Hz, 1H), 3.56 (dd, J=10.8, 6.0 Hz, 1H), 3.35-3.31 (m, 3H), 2.56 (t, J=7.4 Hz, 2H); ¹³C NMR (100 MHz, CD₃OD) δ 175.2, 147.0, 132.6, 131.4, 108.7, 65.5, 57.9, 42.1, 36.1; HRMS (ESI, m/z): calcd for C₁₁H₁₅N₂O₅ ([M−H]⁻): 255.0986, Found: 255.0991.

### Synthesis and Characterization of (S)-2-Amino-N-(3,5-dihydroxy-4-methoxyphenethyl)-3-hydroxypropanamide, JYX008

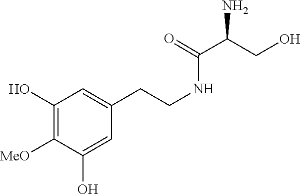 [SMILES: COc1c(O)cc(CCNC(=O)[C@@H](N)CO)cc1O]

This product was prepared according to general procedure A. 1H NMR (400 MHz, CD₃OD) δ 6.21 (s, 2H), 3.73 (s, 3H), 3.64 (dd, J=10.8, 5.2 Hz, 1H), 3.56 (dd, J=10.8, 6.0 Hz, 1H), 3.37-3.30 (m, 3H), 2.58 (t, J=7.4 Hz, 2H); ¹³C NMR (100 MHz, CD₃OD) δ 175.3, 151.6, 136.4, 135.4, 109.0, 65.6, 60.8, 57.9, 41.8, 36.2.

### Synthesis and Characterization of (S)-2-Amino-N-(2,6-dihydroxyphenethyl)-3-hydroxy-propanamide, JYX009

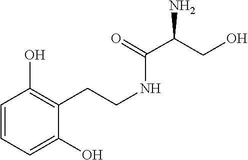 [SMILES: N[C@@H](CO)C(=O)NCCc1c(O)cccc1O]

This product was prepared according to general procedure A. 1H NMR (400 MHz, CD₃OD) δ 6.80 (t, J=8.2 Hz, 1H), 6.28 (d, J=8.0 Hz, 2H), 3.65 (dd, J=10.8, 4.8 Hz, 1H), 3.55 (dd, J=10.8, 6.0 Hz, 1H), 3.36 (td, J=7.0, 2.8 Hz, 2H), 3.32-3.30 (m, 1H), 2.85 (t, J=7.0 Hz, 2H); ¹³C NMR (100 MHz, CD₃OD) δ 175.3, 157.7, 128.2, 113.6, 107.6, 65.4, 58.0, 40.4, 23.5.

### Synthesis and Characterization of (S)-2-Amino-N-(2,5-dihydroxyphenethyl)-3-hydroxy-propanamide, JYX010

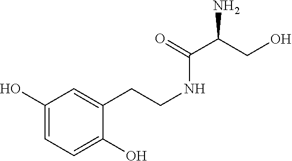 [SMILES: N[C@@H](CO)C(=O)NCCc1cc(O)ccc1O]

This product was prepared according to general procedure A. ¹H NMR (400 MHz, CD₃OD) δ 6.58 (d, J=8.4 Hz, 1H), 6.54 (d, J=2.8 Hz, 1H), 6.47 (dd, J=8.4, 2.8 Hz, 1H), 3.65 (dd, J=10.8, 4.8 Hz, 1H), 3.57 (dd, J=10.8, 6.0 Hz, 1H), 3.40 (td, J=7.2, 1.6 Hz, 2H), 3.34-3.31 (m, 1H), 2.72 (t, J=7.2 Hz, 2H); ¹³C NMR (100 MHz, CD₃OD) δ 175.2, 151.1, 149.4, 127.6, 118.2, 116.8, 114.9, 65.5, 57.9, 40.8, 31.1.

### Synthesis and Characterization of (S)-2-Amino-N-(3,4-dihydroxyphenethyl)-3-hydroxypropanamide, JYX011

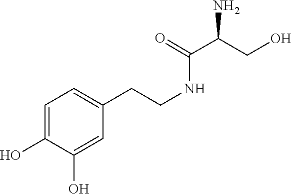 [SMILES: N[C@@H](CO)C(=O)NCCc1ccc(O)c(O)c1]

This product was prepared according to general procedure A. ¹H NMR (400 MHz, CD₃OD) 6.66 (d, J=8.0 Hz, 1H), 6.63 (d, J=2.0 Hz, 1H), 6.51 (dd, J=8.0, 2.0 Hz, 1H), 3.64 (dd, J=10.8, 5.2 Hz, 1H), 3.56 (dd, J=10.8, 6.0 Hz, 1H), 3.37-3.31 (m, 3H), 2.62 (t, J=7.4 Hz, 2H); ¹³C NMR (100 MHz, CD₃OD) δ 175.1, 146.3, 144.8, 132.0, 121.1, 116.9, 116.4, 65.5, 57.8, 42.1, 35.9; HRMS (ESI, m/z): calcd for ([M+H]+). Found:.

### Synthesis and Characterization of (S)-2-Amino-N-(2,3-dihydroxyphenethyl)-3-hydroxy-propanamide, JYX012

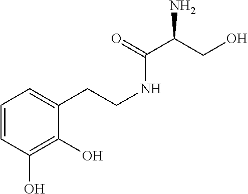 [SMILES: N[C@@H](CO)C(=O)NCCc1cccc(O)c1O]

This product was prepared according to general procedure A. ¹H NMR (400 MHz, CD₃OD) δ 6.65-6.62 (m, 1H), 6.58-6.56 (m, 2H), 3.65 (dd, J=10.8, 4.8 Hz, 1H), 3.57 (dd, J=10.8, 6.4 Hz, 1H), 3.41 (td, J=7.2, 2.8 Hz, 2H), 3.35 (t, J=5.6 Hz, 1H), 2.79 (t, J=7.0 Hz, 2H); ¹³C NMR (100 MHz, CD₃OD) δ 174.9, 146.2, 144.7, 127.1, 122.4, 120.4, 114.6, 65.3, 57.8, 40.8, 30.9.

### Synthesis and Characterization of (S)-2-Amino-N-(3,5-dihydroxyphenethyl)-3-hydroxy-propanamide, JYX013

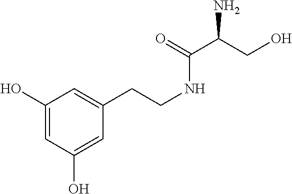 [SMILES: N[C@@H](CO)C(=O)NCCc1cc(O)cc(O)c1]

This product was prepared according to general procedure A. ¹H NMR (400 MHz, CD₃OD) δ 6.14 (d, J=2.4 Hz, 2H), 6.10 (t, J=2.2 Hz, 1H), 3.65 (dd, J=10.8, 5.2 Hz, 1H), 3.57 (dd, J=10.8, 6.0 Hz, 1H), 3.39-3.34 (m, 3H), 2.62 (t, J=7.2 Hz, 2H); ¹³C NMR (100 MHz, CD₃OD) δ 175.0, 159.6, 142.6, 108.3, 101.7, 65.4, 57.8, 41.7, 36.5.

### Synthesis and Characterization of (S)-2-Amino-3-hydroxy-N-(2-hydroxyphenethyl)-propanamide, JYX014

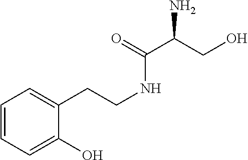 [SMILES: N[C@@H](CO)C(=O)NCCc1ccccc1O]

This product was prepared according to general procedure A. ¹H NMR (400 MHz, CD₃OD) δ 7.06-6.98 (m, 2H), 6.74-6.70 (m, 2H), 3.64 (dd, J=10.8, 4.8 Hz, 1H), 3.56 (dd, J=10.8, 6.0 Hz, 1H), 3.41 (td, J=7.2, 3.2 Hz, 2H), 3.34-3.31 (m, 1H), 2.79 (t, J=7.2 Hz, 2H); ¹³C NMR (100 MHz, CD₃OD) δ 175.1, 156.6, 131.7, 128.7, 126.7, 120.6, 116.0, 65.4, 57.9, 40.7, 31.1.

### Synthesis and Characterization of (S)-2-Amino-3-hydroxy-N-(4-hydroxyphenethyl)-propanamide, JYX015

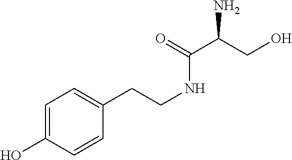 [SMILES: N[C@@H](CO)C(=O)NCCc1ccc(O)cc1]

This product was prepared according to general procedure A. ¹H NMR (400 MHz, CD₃OD) δ 7.03-7.00 (m, 2H), 6.70-6.68 (m, 2H), 3.63 (dd, J=10.8, 4.8 Hz, 1H), 3.56 (dd, J=10.8, 6.4 Hz, 1H), 3.38-3.31 (m, 3H), 2.68 (t, J=7.4 Hz, 2H); ¹³C NMR (100 MHz, CD₃OD) δ 175.2, 157.0, 131.2, 130.8, 116.3, 65.5, 57.8, 42.2, 35.7.

### Synthesis and Characterization of (S)-2-Amino-3-hydroxy-N-(3-hydroxyphenethyl)-propanamide, JYX016

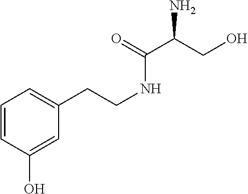 [SMILES: N[C@@H](CO)C(=O)NCCc1cccc(O)c1]

This product was prepared according to general procedure A. ¹H NMR (400 MHz, CD₃OD) δ 7.06 (t, J=7.8 Hz, 1H), 6.68-6.59 (m, 3H), 3.64 (dd, J=10.8, 4.8 Hz, 1H), 3.56 (dd, J=10.8, 6.0 Hz, 1H), 3.39 (td, J=7.6, 1.6 Hz, 2H), 3.33-3.30 (m, 1H), 2.70 (t, J=7.4 Hz, 2H); ¹³C NMR (100 MHz, CD₃OD) δ 175.4, 158.6, 141.9, 130.5, 121.0, 116.6, 114.3, 65.6, 57.9, 41.8, 36.4;.

### Synthesis and Characterization of (S)-2-Amino-3-hydroxy-N-(2-(5-hydroxy-1H-indol-3-yl)ethyl)propanamide, JYX017

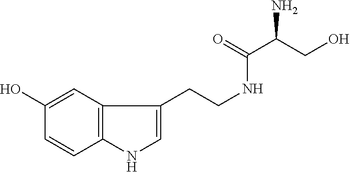 [SMILES: N[C@@H](CO)C(=O)NCCc1c[nH]c2ccc(O)cc12]

This product was prepared according to general procedure A. ¹H HMR (400 MHz, CD₃OD) δ 7.13 (dd, J=8.8, 0.4 Hz, 1H), 7.00 (s, 1H), 6.92 (dd, J=2.4, 0.8 Hz, 1H), 6.64 (dd, J=8.8, 2.0 Hz, 1H), 3.63 (dd, J=10.8, 5.2 Hz, 1H), 3.56 (dd, J=10.8, 6.0 Hz, 1H), 3.46 (t, J=7.2 Hz, 2H), 3.32-3.29 (m, 1H), 2.86 (t, J=7.0 Hz, 2H); ¹³C NMR (100 MHz, CD₃OD) δ 175.4, 151.1, 133.1, 129.4, 124.4, 112.7, 112.4 (2C), 103.5, 65.6, 57.9, 41.2, 26.2.

### Synthesis and Characterization of (S)-2-Oxo-N-(3,4,5-trihydroxyphenethyl)oxazolidine-4-carboxamide, JYX018

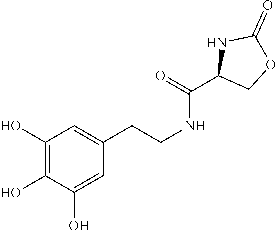 [SMILES: O=C1N[C@H](C(=O)NCCc2cc(O)c(O)c(O)c2)CO1]

This product was prepared according to general procedure A. ¹H NMR (500 MHz, CD₃OD) δ 6.19 (s, 2H), 4.55 (t, J=9.0 Hz, 1H), 4.30 (dd, J=9.5, 5.0 Hz, 1H), 4.21 (dd, J=9.0, 5.5 Hz, 1H), 3.41-3.36 (m, 2H), 2.59 (td, J=7.3, 3.5 Hz, 2H); ¹³C NMR (125 MHz, CD₃OD) δ 172.8, 162.1, 147.1, 132.7, 131.1, 108.7, 69.5, 56.1, 42.2, 35.9

### General Procedure B Exemplified by the Synthesis of 1-(4-Methoxyphenyl)-3-(3,4,5-trihydroxyphenethyl)urea, JYX019

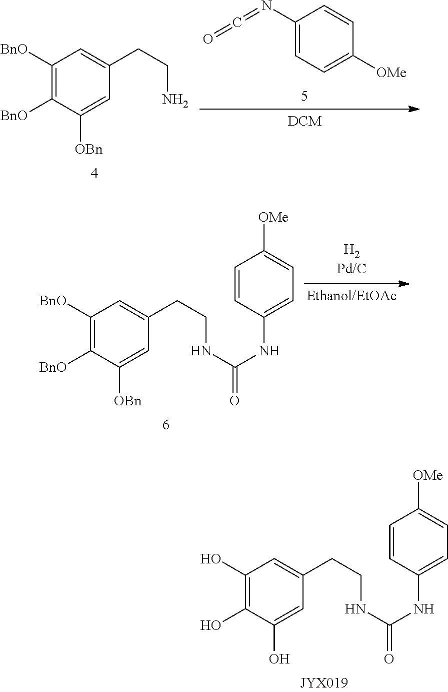 [SMILES: NCCc1cc(OCc2ccccc2)c(OCc2ccccc2)c(OCc2ccccc2)c1 ; COc1ccc(N=C=O)cc1 ; COc1ccc(NC(=O)NCCc2cc(OCc3ccccc3)c(OCc3ccccc3)c(OCc3ccccc3)c2)cc1 ; COc1ccc(NC(=O)NCCc2cc(O)c(O)c(O)c2)cc1]

A mixture of 4 (1.32 g, 3.0 mmol) and 5 (0.54 g, 3.6 mmol) in anhydrous dichloromethane (20.0 ml) was stirred at 21° C. for 12 h under argon. The solvent was removed under vacuum and the resulting residue was purified by flash chromatography on silica gel (dichloromethane/MeOH) to give compound 6 (1.41 g, 80%). Compound 6 (1.41 g, 2.4 mmol) was dissolved in a mixture of ethanol (15.0 ml) and ethyl acetate (15.0 ml) and 5%-Pd/C (255 mg, 0.12 mmol) was then added. The resulting mixture was then stirred at 21° C. for 12 h under hydrogen. The reaction mixture was filtered through a Celite plug, and the solvent was removed under reduced pressure. The resulting residue was purified by flash chromatography on silica gel (dichloromethane/MeOH) to give JYX019 (0.53 g, 69%). ¹H NMR (400 MHz, CD₃OD) δ 7.17-7.15 (m, 2H), 6.80-6.78 (m, 2H), 6.21 (s, 2H), 3.71 (s, 3H), 3.32 (t, J=7.0 Hz, 2H), 2.56 (t, J=7.0 Hz, 2H); ¹³C NMR (100 MHz, CD₃OD) δ 158.8, 157.1, 147.1, 133.6, 132.6, 131.6, 122.9, 115.1, 108.7, 55.9, 42.5, 36.9.

### Synthesis and Characterization of 1-(4-Fluorophenyl)-3-(3,4,5-trihydroxyphenethyl)urea, JYX020

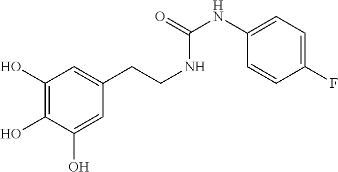 [SMILES: O=C(NCCc1cc(O)c(O)c(O)c1)Nc1ccc(F)cc1]

This product was prepared according to general procedure B. ¹H NMR (400 MHz, CD₃OD) δ 7.29-7.26 (m, 2H), 6.94 (t, J=9.0 Hz, 2H), 6.21 (s, 2H), 3.33 (t, J=7.0 Hz, 2H), 2.57 (t, J=7.0 Hz, 2H); ¹³C NMR (100 MHz, CD₃OD) δ 159.8 (d, J=238.3 Hz), 158.4, 147.1, 137.1 (d, J=2.7 Hz), 132.6, 131.5, 122.1 (d, J=7.6 Hz), 116.1 (d, J=22.6 Hz), 108.7, 42.4, 36.8.

### Synthesis and Characterization of 1-Ethyl-3-(3,4,5-trihydroxyphenethyl)urea, JYX021

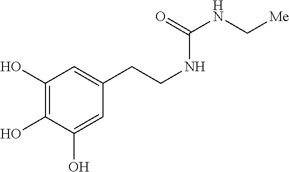 [SMILES: CCNC(=O)NCCc1cc(O)c(O)c(O)c1]

This product was prepared according to general procedure B. 1H NMR (400 MHz, CD₃OD) δ 6.18 (s, 2H), 3.24 (t, J=7.2 Hz, 2H), 3.09 (q, J=7.2 Hz, 2H), 2.51 (t, J=7.4 Hz, 2H), 1.04 (t, J=7.2 Hz, 3H); ¹³C NMR (100 MHz, CD₃OD) δ 161.2, 147.0, 132.5, 131.7, 108.7, 42.7, 37.1, 35.8, 15.7.

General procedure C exemplified by the synthesis of (S)-2-Amino-3-hydroxy-N′-(3,4,5-trihydroxybenzylidene)propanehydrazide, JY022

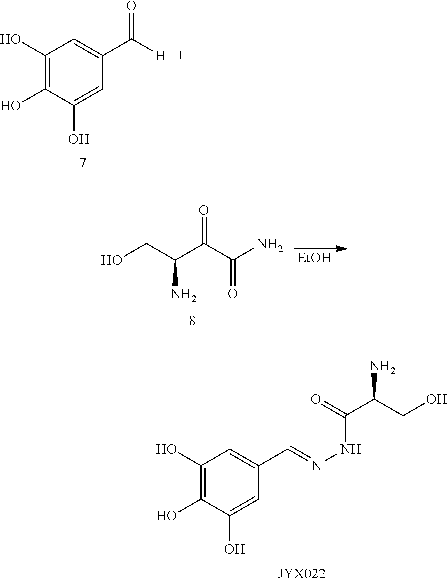

A mixture of 7 (154 mg, 1.0 mmol) and 8 (119 mg, 1.0 mmol) in ethanol (10.0 ml) was stirred at 21° C. for 24 h under argon. The precipitated product was collected by filtration, washed with ethanol and ether, and dried in vacuo to give product JYX022 (222 mg, 87%). ¹H NMR (500 MHz, d₆-DMSO), described as mixture of (E/Z) isomers, a molar ratio ca. 7:3, with the signals of the major isomer marked with asterisks, δ 7.96* (s, 0.7H), 7.68 (s, 0.3H), 6.60* (s, 1.4H), 6.55 (s, 0.6H), 4.08 (t, J=5.3 Hz, 0.3H), 3.59 (dd, J=10.5, 4.5 Hz, 0.3H), 3.50* (dd, J=10.0, 5.5 Hz, 0.7H), 3.49-3.41 (m, 1H), 3.25* (t, J=5.8 Hz, 0.7H); ¹³C NMR (125 MHz, d₆-DMSO), described as mixture of (E/Z) isomers, a molar ratio ca. 7:3, with the signals of the major isomer marked with asterisks, δ 174.8, 170.0*, 148.1*, 146.8, 146.7*, 144.5, 136.3*, 136.2, 125.0*, 124.9, 106.7*, 106.4, 64.9*, 64.3, 56.9*, 53.8; FIRMS (ESI, m/z): calcd for C₁₀H₁₄N₃O₅ ([M+H]⁺): 256.0928, Found: 256.0926.

### Synthesis and Characterization of (S)-2-Amino-3-hydroxy-N′-(4-hydroxy-3,5-dimethoxy-benzylidene)propanehydrazide, JYX023

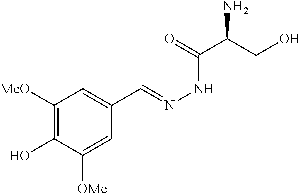 [SMILES: COc1cc(/C=N/NC(=O)[C@@H](N)CO)cc(OC)c1O]

This product was prepared according to general procedure C. 1H NMR (500 MHz, d₆-DMSO), described as mixture of (E/Z) isomers, a molar ratio ca. 7:3, signals of major isomer marked with asterisk, δ 8.12* (s, 0.7H), 7.83 (s, 0.3H), 6.91* (s, 1.4H), 6.89 (s, 0.6H), 4.13 (t, J=5.3 Hz, 0.3H), 3.78* (s, 4.2H), 3.77 (s, 1.8H), 3.60 (dd, J=10.0, 4.5 Hz, 0.3H), 3.51* (dd, J=10.5, 6.0 Hz, 0.7H), 3.47-3.41 (m, 1H), 3.28* (t, J=5.8 Hz, 0.7H); ¹³C NMR (125 MHz, d₆-DMSO), described as mixture of (E/Z) isomers, a molar ratio ca. 7:3, with the signals of the major isomer marked with asterisks, δ 175.3, 170.3*, 148.7, 148.6*, 147.9*, 143.8, 138.3*, 138.1, 125.1*, 125.0, 105.0*, 104.8, 64.9*, 64.7, 56.9*, 56.5*, 53.8; HRMS (ESI, m/z): calcd for C₁₂H₃₈N₃O₅ ([M+H]⁺): 284.1241, Found: 284.1238.

### Synthesis and Characterization of (S)-2-Amino-N-(2-(2,3-dioxo-1,2,3,4-tetrahydroquinoxalin-6-yl)ethyl)-3-hydroxypropanamide, JYX024

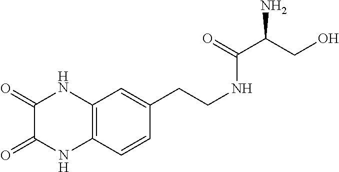 [SMILES: N[C@@H](CO)C(=O)NCCc1ccc2[nH]c(=O)c(=O)[nH]c2c1]

This product was prepared according to general procedure A. ¹H NMR (500 MHz, d₆-DMSO) δ 7.90 (t, J=5.8 Hz, 1H), 7.02 (t, J=8.5 Hz, 1H), 6.92-6.90 (m, 2H), 3.46 (dd, J=10.0, 4.5 Hz, 1H), 3.26-3.20 (m, 3H), 3.14 (t, J=5.5 Hz, 1H), 2.64 (t, J=7.5 Hz, 2H); ¹³C NMR (125 MHz, d₆-DMSO) δ 173.5, 155.8, 155.5, 135.0, 126.0, 124.4, 124.1, 115.6, 115.5, 64.7, 57.4, 49.1, 35.3; HRMS (ESI, m/z): calcd for C₁₃H₁₅N₄O₄ ([M−H]⁻): 291.1099, Found: 291.1098.

### Synthesis and Characterization of (S)-2-Amino-N-(4-amino-3,5-dihydroxyphenethyl)-3-hydroxypropanamide, JYX025

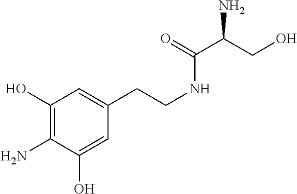 [SMILES: Nc1c(O)cc(CCNC(=O)[C@@H](N)CO)cc1O]

This product was prepared according to general procedure A. ¹H NMR (500 MHz, CD₃OD) δ 6.18 (s, 2H), 3.65 (dd, J=11.0, 5.0 Hz, 1H), 3.57 (dd, J=11.0, 6.0 Hz, 1H), 3.36-3.32 (m, 3H), 2.57 (t, J=7.3 Hz, 2H); ¹³C NMR (125 MHz, CD₃OD) δ 175.4, 147.6, 131.0, 121.4, 108.4, 65.6, 57.9, 42.1, 36.1; HRMS (ESI, m/z): calcd for C₁₁H₁₈N₃O₄ ([M+H]⁺): 256.1292, Found: 256.1287.

### Synthesis and Characterization of (S)-2-Amino-N′-(2,3-dihydroxybenzylidene)-3-hydroxy-propanehydrazide, JYX026

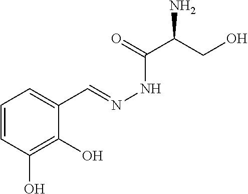 [SMILES: N[C@@H](CO)C(=O)N/N=C/c1cccc(O)c1O]

This product was prepared according to general procedure C. 1B NMR (500 MHz, CD₃OD) δ 8.31 (s, 1H), 6.87-6.85 (m, 2H), 6.75 (dd, J=8.0, 7.0 Hz, 1H), 3.74 (d, J=5.5 Hz, 2H), 3.49 (t, J=5.3 Hz, 1H); ¹³C NMR (125 MHz, CD₃OD) δ 171.8, 151.9, 147.5, 146.8, 122.5, 120.5, 119.4, 118.7, 65.5, 57.1; FIRMS (ESI, m/z): calcd for C₁₀H₁₄N₃O₄ ([M+H]⁺): 240.0979, Found: 240.0978.

### Synthesis and Characterization of (S)-2-Amino-N′-(3,4-dihydroxybenzylidene)-3-hydroxypropanehydrazide, JYX027

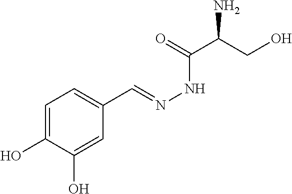 [SMILES: N[C@@H](CO)C(=O)N/N=C/c1ccc(O)c(O)c1]

This product was prepared according to general procedure C. ¹H NMR (500 MHz, CD₃OD), described as mixture of (E/Z) isomers, a molar ratio ca. 7:3, signals of major isomer marked with asterisk, δ 7.99* (s, 0.7H), 7.78 (s, 0.3H), 7.29* (d, J=1.5 Hz, 0.7H), 7.18 (d, J=2.0 Hz, 0.3H), 7.00* (dd, J=8.0, 1.5 Hz, 0.7H), 6.93 (dd, J=8.0, 1.5 Hz, 0.3H), 6.77 (d, J=8.0 Hz, 1H), 4.37 (t, J=5.0 Hz, 0.3H), 3.87 (dd, J=11.0, 4.0 Hz, 0.3H), 3.74-3.70* (m, 1.4H), 3.60 (dd, J=14.0, 7.0 Hz, 0.3H), 3.46* (t, J=5.8 Hz, 0.7H); ¹³C NMR (125 MHz, CD₃OD), described as mixture of (E/Z) isomers, a molar ratio ca. 7:3, with the signals of the major isomer marked with asterisks, δ 175.4, 172.1*, 150.8*, 149.9*, 149.5, 147.0, 146.93*, 146.91, 127.4, 127.1*, 122.7*, 121.9, 116.3, 116.2*, 114.1*, 113.7, 65.5*, 64.7, 57.1*, 54.8; HRMS (ESI, m/z): calcd for C₁₀H₁₄N₃O₄ ([M+H]⁺): 240.0979, Found: 240.0976.

### Synthesis and Characterization of (S)-2-Amino-N′-(2,5-dihydroxybenzylidene)-3-hydroxypropanehydrazide, JYX028

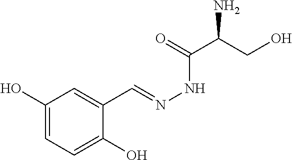 [SMILES: N[C@@H](CO)C(=O)N/N=C/c1cc(O)ccc1O]

This product was prepared according to general procedure C. ¹H NMR (500 MHz, CD₃OD) δ 8.28 (s, 1H), 6.83 (d, J=2.5 Hz, 1.0 Hz), 6.76 (d, J=3.0 Hz, 1H), 6.75 (s, 1H), 3.73 (d, J=5.5 Hz, 2H), 3.48 (t, J=5.8 Hz, 1H); ¹³C NMR (125 MHz, CD₃OD) δ 171.9, 152.5, 151.2, 151.1, 120.5, 119.5, 118.3, 116.4, 65.5, 57.1; HRMS (ESI, m/z): calcd for C₁₀H₁₆N₃O₄ ([M+H]⁺): 240.0979, Found: 240.0978.

### Synthesis and Characterization of (S)-2-Amino-N′-(3,4-dihydroxy-5-methoxybenzylidene)-3-hydroxypropanehydrazide, JYX029

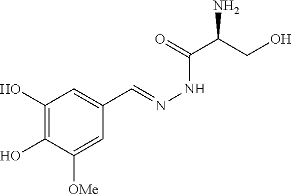 [SMILES: COc1cc(/C=N/NC(=O)[C@@H](N)CO)cc(O)c1O]

This product was prepared according to general procedure C. ¹H NMR (500 MHz, d₆-DMSO), described as mixture of (E/Z) isomers, a molar ratio ca. 7:3, signals of major isomer marked with asterisk, δ 8.04* (s, 0.7H), 7.76 (s, 0.3H), 6.76-6.75 (m, 1H), 6.74* (d, J=1.5 Hz, 0.7H), 6.69 (d, J=1.5 Hz, 0.3H), 4.11 (dd, J=6.0, 5.0 Hz, 0.3H), 3.76* (s, 2.1H), 3.75 (s, 0.9H), 3.73-3.71 (m, 0.3H), 3.60 (dd, J=10.5, 5.0 Hz, 0.3H), 3.50* (dd, J=10.5, 5.5 Hz, 0.7H), 3.45* (dd, J=10.5, 6.0 Hz, 0.7H), 3.26* (t, J=5.8 Hz, 0.7H); ¹³C NMR (125 MHz, d₆-DMSO), described as mixture of (E/Z) isomers, a molar ratio ca. 7:3, with the signals of the major isomer marked with asterisks, δ 175.1, 170.1*, 149.0*, 148.9, 148.0*, 146.5, 146.4*, 144.1, 137.2*, 137.0, 125.1*, 125.0, 108.8*, 107.7, 103.4, 102.8*, 64.9*, 64.5, 56.9*, 56.37, 56.35*, 53.8; FIRMS (ESI, m/z): calcd for C₁₁H₁₆N₃O₅ ([M+H]⁺): 270.1084, Found: 270.1082.

### Synthesis and Characterization of (S)-2-Amino-N′-(3-fluoro-2,4-dihydroxybenzylidene)-3-hydroxypropanehydrazide, JYX030

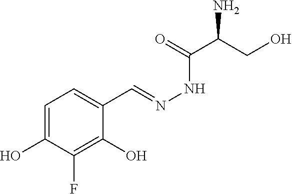 [SMILES: N[C@@H](CO)C(=O)N/N=C/c1ccc(O)c(F)c1O]

This product was prepared according to general procedure C. 1H NMR (500 MHz, CD₃OD) δ 8.24 (s, 1H), 6.98 (dd, J=7.8, 1.5 Hz, 1H), 6.44 (t, J=8.0 Hz, 1H), 3.75 (d, J=5.0 Hz, 2H), 3.52 (t, J=5.5 Hz, 1H); ¹³C NMR (125 MHz, CD₃OD) δ 171.0, 151.7 (d, J=3.1 Hz), 150.6 (d, J=10.5 Hz), 149.1 (d, J=10.6 Hz). 140.0 (d, J=235.6 Hz), 126.7 (d, J=3.9 Hz,), 112.8 (d, J=1.9 Hz), 109.8 (d, J=1.3 Hz), 65.2, 57.0; HRMS (ESI, m/z): calcd for C₁₀H₁₃FN₃O₄ ([M+H]⁺): 258.0885, Found: 258.0876.

### Synthesis and Characterization of (S)-2-Amino-N′-(2,4-dihydroxy-3-(hydroxymethyl)-benzylidene)-3-hydroxypropanehydrazide, JYX031

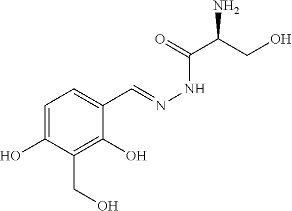 [SMILES: N[C@@H](CO)C(=O)N/N=C/c1ccc(O)c(CO)c1O]

This product was prepared according to general procedure C. 1H NMR (500 MHz, CD₃OD) δ 8.21 (s, 1H), 7.06 (d, J=10.5 Hz, 1H), 6.39 (d, J=10.5 Hz, 1H), 4.76 (s, 2H), 3.71 (d, J=6.8 Hz, 2H), 3.45 (t, J=6.8 Hz, 1H); ¹³C NMR (125 MHz, d₆-DMSO) δ 170.0, 159.6, 158.5, 150.1, 131.3, 114.9, 110.4, 107.8, 64.7, 56.5, 53.0; HRMS (ESI, m/z): calcd for([M+H]⁺):, Found:.

### Synthesis and Characterization of N-(6-((2-(L-Seryl)hydrazineylidene)methyl)-2,3-dihydroxyphenyl)acetamide, JYX032

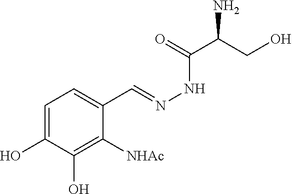 [SMILES: CC(=O)Nc1c(/C=N/NC(=O)[C@@H](N)CO)ccc(O)c1O]

This product was prepared according to general procedure C. 1H NMR (500 MHz, d₆-DMSO), described as mixture of (E/Z) isomers, a molar ratio ca. 8:2, signals of major isomer marked with asterisk, δ 8.14* (s, 0.8H), 7.84 (s, 0.2H), 7.14 (d, J=8.5 Hz, 0.2H), 7.05* (d, J=6.4 Hz, 0.8H), 6.70 (d, J=8.0 Hz, 1H), 3.61-3.58 (m, 0.4H), 3.51-3.47* (m, 1.6H), 3.28 (t, J=5.3 Hz, 1H), 2.18* (s, 2.4H), 2.03 (s, 0.6H); ¹³C NMR (125 MHz, d₆-DMSO), described as mixture of (E/Z) isomers, a molar ratio ca. 8:2, with the signals of the major isomer marked with asterisks, δ 173.5, 170.3*, 169.5*, 169.4, 148.3*, 147.3, 146.1*, 141.9, 140.4, 139.1*, 125.1*, 121.8, 119.4*, 119.2*, 116.0, 113.5, 112.9*, 112.6, 63.8*, 63.1, 55.7*, 52.7, 22.8*, 22.5; HRMS (ESI, m/z): calcd for C₁₂F₁₅N₄O₅ ([M−H]⁻): 295.1048, Found: 295.1049.

### General Procedure D Exemplified by the Synthesis of (S)-2-Amino-3-hydroxy-1-(6,7,8-trihydroxy-3,4-dihydroisoquinolin-2(1H)-yl)propan-1-one, JYX033

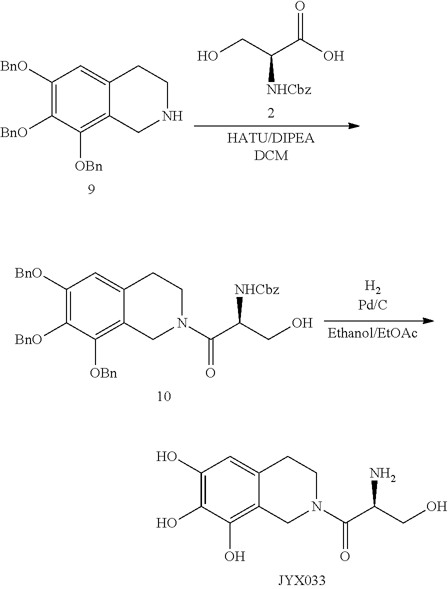 [SMILES: c1ccc(COc2cc3c(c(OCc4ccccc4)c2OCc2ccccc2)CNCC3)cc1 ; O=C(N[C@@H](CO)C(=O)O)OCc1ccccc1 ; O=C(N[C@@H](CO)C(=O)N1CCc2cc(OCc3ccccc3)c(OCc3ccccc3)c(OCc3ccccc3)c2C1)OCc1ccccc1 ; N[C@@H](CO)C(=O)N1CCc2cc(O)c(O)c(O)c2C1]

To a solution of 9 (0.84 g, 1.86 mmol), 2 (0.45 g, 1.86 mmol) and HATU (0.71 g, 1.86 mmol) in anhydrous dichloromethane (20.0 ml) was dropwise added diisopropylethylamine (DIPEA, 0.65 ml) at 0° C. under argon. After stirring at 0° C. for 15 min, the mixture was warmed to 21° C. and stirred for 2 h. The mixture was diluted with dichloromethane, washed with saturated sodium bicarbonate, dried over anhydrous sodium sulfate, and concentrated under reduced pressure. The resulting residue was purified by flash choreography on silica gel (dichloro-methane/MeOH) to give the compound 10 (1.08 g, 86%). Compound 10 (1.08 g, 1.60 mmol) was dissolved in a mixture of ethanol (10.0 ml) and ethyl acetate (10.0 ml) and 5%-Pd/C (170 mg, 0.093 mmol) was then added. The resulting mixture was then stirred at 21° C. for 12 h under hydrogen. The reaction mixture was filtered through a Celite plug, and the solvent was removed under reduced pressure. The resulting residue was purified by flash chromatography on silica gel (dichloromethane/MeOH) to give JYX033 (150 mg, 35%). 1H NMR (500 MHz, CD₃OD), described as mixture of rotamers, a molar ratio ca. 1:1, δ 6.16 (s, 1H), 4.65-4.51 (m, 2H), 4.04 (t, J=6.0 Hz, 0.5H), 3.99 (t, J=6.0 Hz, 0.5H), 3.79-3.65 (m, 3H), 3.59-3.51 (m, 1H), 2.74 (br s, 1H), 2.65 (t, J=5.8 Hz, 1H); ¹³C NMR (125 MHz, CD₃OD), described as mixture of rotamers, a molar ratio ca. 1:1, δ 173.8, 173.5, 146.2, 145.9, 144.1, 143.8, 132.5, 132.4, 126.8, 126.3, 112.9, 112.6, 107.4, 107.2, 65.8, 65.5, 54.2, 53.8, 44.7, 44.0, 42.0, 41.8, 30.1, 28.9; FIRMS (ESI, m/z): calcd for([M+H]⁺). Found:.

### General Procedure E Exemplified by the Synthesis of (S)-2-Amino-3-hydroxy-N-(2′,3′,4′-trihydroxy-[1,1′-biphenyl]-2-yl)propanamide, JYX034

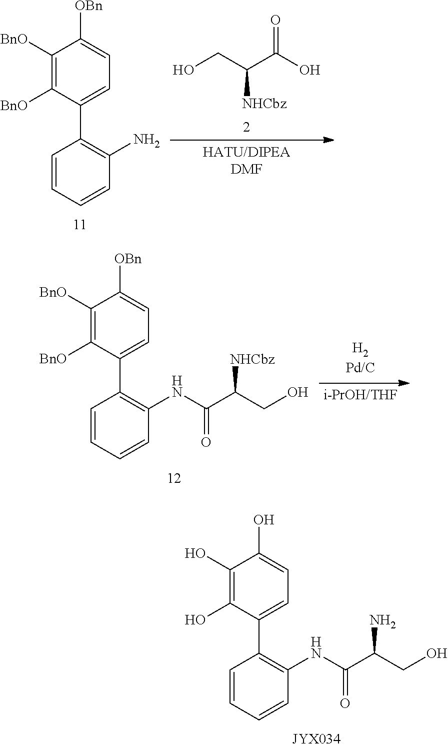 [SMILES: Nc1ccccc1-c1ccc(OCc2ccccc2)c(OCc2ccccc2)c1OCc1ccccc1 ; O=C(N[C@@H](CO)C(=O)O)OCc1ccccc1 ; O=C(N[C@@H](CO)C(=O)Nc1ccccc1-c1ccc(OCc2ccccc2)c(OCc2ccccc2)c1OCc1ccccc1)OCc1ccccc1 ; N[C@@H](CO)C(=O)Nc1ccccc1-c1ccc(O)c(O)c1O]

To a solution of 11 (1.95 g, 4.0 mmol), 12 (1.44 g, 6.0 mmol) and HATU (2.28 g, 6.0 mmol) in anhydrous DMF (30.0 ml) was added diisopropylethylamine (DIPEA, 1.4 ml) under argon. The mixture was then heated to 60° C. and stirred for 5 h. The mixture was cooled to 21° C. and the solvent was removed under reduced pressure. The residue was dissolved in ethyl acetate, washed with saturated sodium bicarbonate and brine, dried over anhydrous sodium sulfate, and concentrated under reduced pressure. The resulting residue was purified by flash choreography on silica gel (dichloromethane/MeOH) to give the compound 12 (1.77 g, 63%). Compound 12 (1.77 g, 2.5 mmol) was dissolved in a mixture of isopropanol (15.0 ml) and THY (15.0 ml) and 5%-Pd/C (266 mg, 0.125 mmol) was then added. The resulting mixture was then stirred at 21° C. for 12 h under hydrogen. The reaction mixture was filtered through a Celite plug, and the filtrate was concentrated to about 5.0 ml under reduced pressure. The precipitated product was collected by filtration, washed with isopropanol and ether, and dried in vacuo to give product JYX034 (298 mg, 39%). ¹H NMR (500 MHz, CD₃OD), described as mixture of rotamers, a molar ratio ca. 8:2, with the signals of the major isomer marked with asterisks, δ 7.94* (d, J=8.0 Hz, 0.8H), 7.86 (d, J=8.0 Hz, 0.2H), 7.29 (td, J=7.8, 1.5 Hz, 1H), 7.24 (dd, J=7.5, 1.5 Hz, 1H), 7.17 (qd, J=7.8, 1.0 Hz, 1H), 6.51-6.48 (m, 1H), 6.46-6.44 (m, 1H), 4.41-4.40 (m, 0.2H), 3.93-3.90 (m, 0.2H), 3.70-3.69 (m, 1H), 3.64-3.61* (m, 0.8H), 3.41* (br s, 0.8H); ¹³C NMR (125 MHz, CD₃OD), described as mixture of rotamers, a molar ratio ca. 8:2, with the signals of the major isomer marked with asterisks, δ 173.8*, 170.5, 147.2, 147.1*, 144.5*, 144.3, 136.6*, 136.1, 134.6*, 134.5, 133.8, 133.2*, 132.3, 132.2*, 128.3*, 128.2, 126.4, 125.9*, 124.5, 123.7*, 122.6, 122.3*, 119.4, 119.0*, 108.9, 108.7*, 66.9*, 65.1*, 65.0, 62.7; HRMS (ESI, m/z): calcd for([M+H]+). Found:.

### Synthesis and Characterization of (S)-2-Amino-3-hydroxy-N-(2′,3′,4′-trihydroxy-[1,1′-biphenyl]-3-yl)propanamide, JYX035

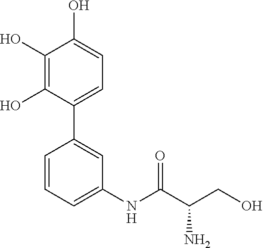 [SMILES: N[C@@H](CO)C(=O)Nc1cccc(-c2ccc(O)c(O)c2O)c1]

This product was prepared according to general procedure E. ¹H NMR (500 MHz, CD₃OD) δ 7.69-7.68 (m, 1H), 7.54-7.52 (m, 1H), 7.30-7.28 (m, 2H), 6.63 (d, J=8.5 Hz, 1H), 6.41 (d, J=8.5 Hz, 1H), 3.83-3.77 (m, 2H), 3.64 (t, J=5.5 Hz, 1H); ¹³C NMR (125 MHz, CD₃OD) 172.7, 146.6, 144.6, 141.3, 138.9, 134.5, 129.3, 126.5, 122.2, 122.0, 121.5, 119.2, 108.3, 64.9, 58.1; HRMS (ESI, m/z): calcd for C₁₅H₁₅N₂O₅ ([M−H]⁻): 303.0986, Found: 303.0974.

### Synthesis and Characterization of (S)-2-Amino-3-hydroxy-N-(2′,3′,4′-trihydroxy-[1,1′-biphenyl]-4-yl)propanamide, JYX036

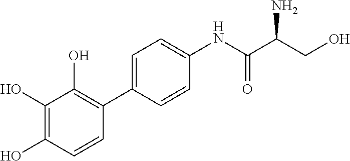 [SMILES: N[C@@H](CO)C(=O)Nc1ccc(-c2ccc(O)c(O)c2O)cc1]

This product was prepared according to general procedure E. ¹H NMR (500 MHz, CD₃OD) δ 7.57 (d, J=8.5 Hz, 2H), 7.49 (d, J=9.0 Hz, 2H), 6.62 (d, J=8.5 Hz, 1H), 6.40 (d, J=8.5 Hz, 1H), 3.81-3.78 (m, 2H), 3.62 (t, J=5.5 Hz, 1H); ¹³C NMR (125 MHz, CD₃OD) δ 172.8, 146.4, 144.6, 137.3, 136.8, 134.5, 130.5, 121.8, 121.3, 120.9, 108.3, 65.0, 58.2; HRMS (ESI, m/z): calcd for C₁₅H₁₅N₂O₅ ([M−H]⁻): 303.0986, Found: 303.0973.

### Synthesis and Characterization of N-(4-Fluorophenyl)-3-(2,3,4-trihydroxyphenyl)-1H-pyrazole-1-carboxamide, JYX037

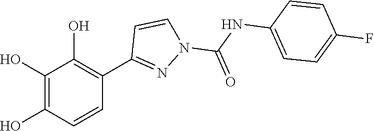 [SMILES: O=C(Nc1ccc(F)cc1)n1ccc(-c2ccc(O)c(O)c2O)n1]

This product was prepared according to general procedure B. ¹H NMR (500 MHz, CD₃OD) δ 8.35 (d, J=3.0 Hz, 1H), 7.68-7.65 (m, 2H), 7.14-7.10 (m, 3H), 6.91 (d, J=3.0 Hz, 1H), 6.46 (d, J=8.5 Hz, 1H); ¹³C NMR (125 MHz, CD₃OD) δ 161.3 (d, J=241.3 Hz), 156.3, 149.4, 148.3, 146.2, 134.7 (d, J=2.9 Hz), 134.4, 131.1, 124.6 (d, J=8.1 Hz), 119.7, 116.4 (d, J=22.8 Hz), 110.2, 108.7, 107.6; HRMS (ESI, m/z): calcd for C₁₆H₁₁FN₃O₄ ([M−H]⁻): 328.0739, Found: 328.0735.

### Synthesis and Characterization of (S)-2-Amino-3-hydroxy-1-(4-(2,3,4-trihydroxyphenyl)-piperazin-1-yl)propan-1-one, JYX038

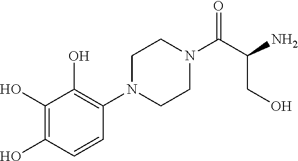 [SMILES: N[C@@H](CO)C(=O)N1CCN(c2ccc(O)c(O)c2O)CC1]

This product was prepared according to general procedure D. ¹H NMR (500 MHz, CD₃OD) δ 6.42 (d, J=9.0 Hz, 1H), 6.28 (d, J=8.5 Hz, 1H), 3.95 (t, J=6.0 Hz, 1H), 3.78-3.73 (m, 4H), 3.65 (dd, J=11.0, 6.0 Hz, 1H), 3.56 (dd, J=10.5, 6.0 Hz, 1H), 2.86-2.81 (m, 4H); ¹³C NMR (125 MHz, CD₃OD) δ 173.4, 144.6, 141.4, 134.3, 133.5, 111.7, 107.0, 65.9, 53.8, 53.3, 53.2, 47.1, 43.7; HRMS (ESI, m/z): calcd for C₁₃H₁₈N₃O₅ ([M−H]⁻): 296.1252, Found: 296.1247.

### General Procedure F Exemplified by the Synthesis of N-Hydroxy-2-(2-(3,4,5-trihydroxyphenyl)acetamido)acetamide, JYX039

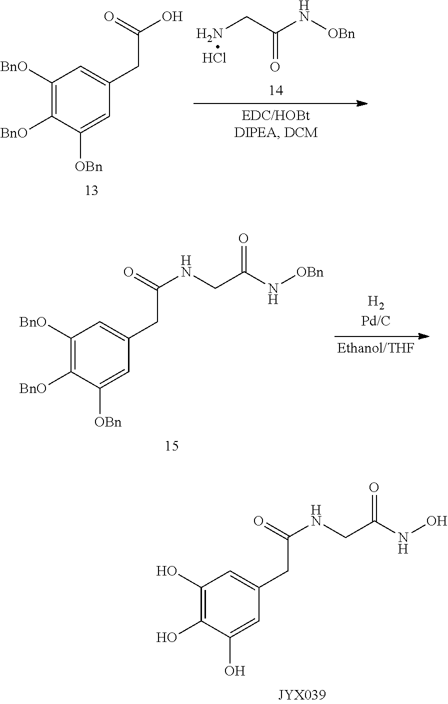 [SMILES: O=C(O)Cc1cc(OCc2ccccc2)c(OCc2ccccc2)c(OCc2ccccc2)c1 ; Cl ; NCC(=O)NOCc1ccccc1 ; O=C(Cc1cc(OCc2ccccc2)c(OCc2ccccc2)c(OCc2ccccc2)c1)NCC(=O)NOCc1ccccc1 ; O=C(CNC(=O)Cc1cc(O)c(O)c(O)c1)NO]

To a solution of 9 (1.3 g, 2.86 mmol), 10 (0.62 g, 2.86 mmol) and 1-Hydroxybenzotriazole hydrate (HOBT, 0.66 g, 4.3 mmol) in anhydrous dichloromethane (20.0 ml) was added diisopropylethylamine (DIPEA, 1.0 ml) at 0° C. under argon. After stirring at 0° C. for 15 minutes, a suspension of EDCHCl (0.83 g, 4.3 mmol) in dichloromethane (5.0 ml) was added dropwise. Then the mixture was warmed to 21° C. and stirred for 16 h. The mixture was diluted with dichloromethane, washed with saturated sodium bicarbonate, dried over anhydrous sodium sulfate, and concentrated under reduced pressure. The resulting residue was purified by flash choreography on silica gel (dichloromethane/MeOH) to give the compound 11 (1.31 g, 74%). Compound 11 (1.31 g, 2.12 mmol) was dissolved in a mixture of ethanol (15.0 ml) and anhydrous THF (15.0 ml) and 5%-Pd/C (226 mg, 0.106 mmol) was then added. The resulting mixture was stirred at 21° C. for 12 h under hydrogen. The reaction mixture was filtered through a Celite plug, and the solvent was removed under reduced pressure. The resulting residue was purified by flash chromatography on silica gel (dichloromethane/MeOH) to give JYX039 (261 mg, 48%). ¹H NMR (500 MHz, CD₃OD) δ 6.28 (s, 2H), 3.76 (s, 2H), 3.34 (s, 2H); ¹³C NMR (125 MHz, CD₃OD) δ 175.2, 168.7, 147.1, 133.3, 127.1, 109.2, 43.3, 41.4; HRMS (ESI, m/z): calcd for C₁H₁₁N₂O₆ ([M−H]⁻): 255.0623, Found: 255.0620.

### Synthesis and Characterization of 2-(2,4-Dihydroxy-3-methoxyphenyl)-N-(2-(hydroxy-amino)-2-oxoethyl)acetamide, JYX040

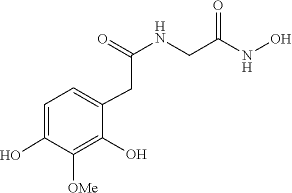 [SMILES: COc1c(O)ccc(CC(=O)NCC(=O)NO)c1O]

This product was prepared according to general procedure F. ¹H NMR (500 MHz, CD₃OD) δ 6.71 (d, J=8.0 Hz, 1H), 6.32 (d, J=8.5 Hz, 1H), 3.78 (s, 3H), 3.47 (s, 2H), 3.34 (s, 2H); ¹³C NMR (125 MHz, CD₃OD) δ 175.6, 168.6, 151.1, 150.0, 137.2, 126.6, 115.0, 108.5, 60.8, 41.6, 38.5; HRMS (ESI, m/z): calcd for C₁₁H₃N₂O₆ ([M−H]⁻): 269.0779, Found: 269.0778.

### Synthesis and Characterization of 2-(3-Fluoro-2,4-dihydroxyphenyl)-N-(2-(hydroxy-amino)-2-oxoethyl)acetamide, JYX041

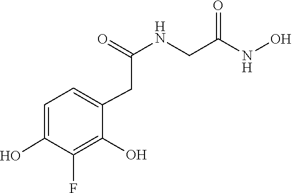 [SMILES: O=C(CNC(=O)Cc1ccc(O)c(F)c1O)NO]

This product was prepared according to general procedure F. ¹H NMR (500 MHz, CD₃OD) δ 6.73 (dd, J=8.5, 2.0 Hz, 1H), 6.37 (t, J=8.3 Hz, 1H), 3.78 (s, 2H), 3.50 (s, 2H); ¹³C NMR (125 MHz, CD₃OD) δ 175.3, 168.6, 146.2 (d, J=10.9 Hz), 145.2 (d, J=12.1 Hz), 143.1 (d, J=232.3 Hz), 126.0 (d, J=4.1 Hz), 116.1, 109.0 (d, J=0.9 Hz), 41.6, 38.1 (d, J=2.1 Hz); HRMS (ESI, m/z): calcd for C₁₀H₁₀FN₂O₅ ([M−H]⁻): 257.0579, Found: 257.0570.

### Synthesis and Characterization of N-Hydroxy-2-(2-(2,3,4-trimethoxyphenyl)acetamido)-acetamide, JYX042

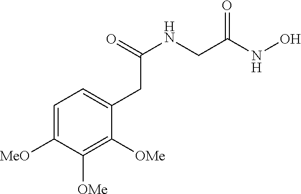 [SMILES: COc1ccc(CC(=O)NCC(=O)NO)c(OC)c1OC]

This product was prepared according to general procedure F. ¹H NMR (500 MHz, d₆-DMSO) δ 10.46 (s, 1H), 8.79 (d, J=1.5 Hz, 1H), 8.05 (t, J=5.8 Hz, 1H), 6.88 (d, J=8.5 Hz, 1H), 6.70 (d, J=8.5 Hz, 1H), 3.74 (s, 3H), 3.71 (s, 3H), 3.70 (s, 3H), 3.59 (d, J=6.0 Hz, 2H), 3.36 (s, 2H); ¹³C NMR (125 MHz, d₆-DMSO) δ 171.3, 166.4, 152.8, 152.0, 142.2, 125.4, 122.6, 108.1, 61.1, 60.8, 56.3, 40.6, 36.7; HRMS (ESI, m/z): calcd for C₁₃H₁₇N₂O₆ ([M−H]⁻): 297.1092, Found: 297.1085.

### Synthesis and Characterization of N-Hydroxy-2-(2-(2,3,4-trifluorophenyl)acetamido)-acetamide, JYX043

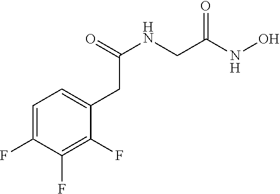 [SMILES: O=C(CNC(=O)Cc1ccc(F)c(F)c1F)NO]

This product was prepared according to general procedure F. ¹H NMR (500 MHz, d₆-DMSO) δ 10.53 (s, 1H), 8.82 (s, 1H), 8.37 (t, J=5.5 Hz, 1H), 7.24-7.21 (m, 1H), 7.20-7.16 (m, 1H), 3.60 (d, J=6.0 Hz, 2H), 3.56 (s, 2H); ¹³C NMR (125 MHz, d₆-DMSO) δ 169.3, 166.2, 150.6 (td, J=9.8, 3.0 Hz), 148.6 (td, J=10.9, 3.0 Hz), 139.3 (dt, J=246.0, 15.6 Hz), 126.4 (m), 121.9 (m), 112.6 (dd, J=16.8, 3.5 Hz), 42.5, 35.0; HRMS (ESL m/z): calcd for C₁₀H₈F₃N₂O₃ ([M−H]⁻): 261.0493, Found: 261.0494.

### Synthesis and Characterization of N-Hydroxy-2-(2-(3-hydroxy-2,4-dimethoxyphenyl)-acetamido)acetamide, JYX044

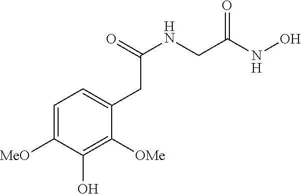 [SMILES: COc1ccc(CC(=O)NCC(=O)NO)c(OC)c1O]

This product was prepared according to general procedure F. ¹H NMR (500 MHz, CD₃OD) δ 6.67 (br s, 2H), 3.83 (s, 6H), 3.78 (s, 2H), 3.50 (s, 2H); ¹³C NMR (125 MHz, CD₃OD) δ 175.1, 168.7, 149.8, 147.4, 140.8, 122.3, 121.5, 108.1, 60.7, 56.7, 41.6, 38.3; HRMS (ESI, m/z): calcd for C₁₂H₁₇N₂O₆ ([M+H]⁺): 285.1081, Found: 285.1075.

### Synthesis and Characterization of (S)-2-Amino-3-hydroxy-N-(3-hydroxy-2,4-dimethoxy-phenethyl)propanamide, JYX045

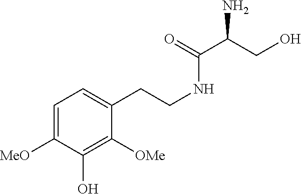 [SMILES: COc1ccc(CCNC(=O)[C@@H](N)CO)c(OC)c1O]

This product was prepared according to general procedure A. ¹H NMR (500 MHz, CD₃OD) δ 6.65 (d, J=8.5 Hz, 1H), 6.62 (d, J=8.5 Hz, 1H), 3.82 (s, 3H), 3.81 (s, 3H), 3.67 (dd, J=10.5, 5.0 Hz, 1H), 3.58 (dd, J=10.5, 6.0 Hz, 1H), 3.40-3.34 (m, 3H), 2.75 (t, J=7.3 Hz, 2H); ¹³C NMR (125 MHz, CD₃OD) δ 175.0, 149.1, 147.6, 140.7, 126.0, 120.7, 108.2, 65.3, 60.9, 57.9, 56.7, 41.5, 30.6; HRMS (ESI, m/z): calcd for C₁₃H₂₁N₂O₅ ([M+H]⁺): 285.1445, Found: 285.1437.

### Synthesis and Characterization of (S)-2-Amino-3-hydroxy-N′-(3-hydroxy-2,4-dimethoxy-benzylidene)propanehydrazide, JYX046

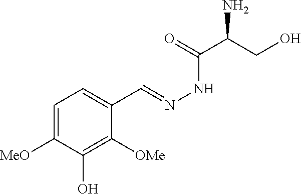 [SMILES: COc1ccc(/C=N/NC(=O)[C@@H](N)CO)c(OC)c1O]

This product was prepared according to general procedure C. ¹H NMR (500 MHz, CD₃OD), described as mixture of (E/Z) isomers, a molar ratio ca. 7:3, signals of major isomer marked with asterisk, δ 8.42* (s, 0.7H), 8.19 (s, 0.3H), 7.54* (d, J=8.5 Hz, 0.7H), 7.34 (d, J=9.0 Hz, 0.3H), 6.79-6.77 (m, 1H), 4.45 (t, J=5.0 Hz, 0.3H), 3.92 (dd, J=11.0, 4.5 Hz, 0.3H), 3.88 (s, 3H), 3.85* (s, 2.1H), 3.84 (s, 0.9H), 3.77 (dd, J=10.5, 5.0 Hz, 0.3H), 3.75-3.73* (m, 1.4H), 3.49* (t, J=5.8 Hz, 0.7H); ¹³C NMR (125 MHz, CD₃OD), described as mixture of (E/Z) isomers, a molar ratio ca. 7:3, with the signals of the major isomer marked with asterisks, δ 174.2, 171.8*, 152.4*, 152.1, 149.1*, 148.9, 146.6*, 143.0, 140.6, 140.4*, 121.5, 121.3*, 118.0*, 117.3, 108.7, 108.6*, 65.4*, 64.0, 61.9*, 61.8, 57.0*, 56.70, 56.68*, 55.0; HRMS (ESI, m/z): calcd for C₁₂H₁₈N₃O₅ ([M+H]⁺): 284.1241, Found: 284.1234.

### Synthesis and Characterization of (S)-2-Amino-3-hydroxy-N′-(pyridin-2-ylmethylene)-propanehydrazide, JYX047

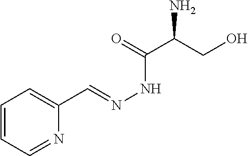 [SMILES: N[C@@H](CO)C(=O)N/N=C/c1ccccn1]

This product was prepared according to general procedure C. ¹H NMR (500 MHz, CD₃OD), described as mixture of (E/Z) isomers, a molar ratio ca. 7:3, signals of major isomer marked with asterisk, δ 8.55 (d, J=5.0 Hz, 1H), 8.21 (s, 1H), 8.20 (s, 0.3H), 7.99-7.97* (m, 0.7H), 7.88-7.85 (m, 1H), 7.42-7.40 (m, 1H), 4.43 (t, J=5.0 Hz, 0.3H), 3.86 (dd, J=11.0, 4.0 Hz, 0.3H), 3.76-3.73 (m, 1.7H), 3.52* (t, J=5.3 Hz, 0.7H); ¹³C HMR (125 MHz, CD₃OD), described as mixture of (E/Z) isomers, a molar ratio ca. 7:3, with the signals of the major isomer marked with asterisks, δ 176.4, 173.1*, 154.4*, 154.3, 150.3, 150.2*, 148.9*, 144.9, 138.7, 138.6*, 126.1*, 125.8, 122.3*, 122.0, 65.5*, 65.1, 57.2*, 54.7; HRMS (ESI, m/z): calcd for C₉H₁₃N₄O₂ ([M+H]⁺): 209.1033, Found: 209.1027.

### Synthesis and Characterization of (S)-2-Amino-3-hydroxy-N′-(pyridin-3-ylmethylene)-propanehydrazide, JYX048

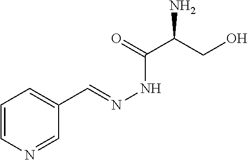 [SMILES: N[C@@H](CO)C(=O)N/N=C/c1cccnc1]

This product was prepared according to general procedure C. ¹H NMR (500 MHz, CD₃OD), described as mixture of (E/Z) isomers, a molar ratio ca. 7:3, with the signals of the major isomer marked with asterisks, δ 8.84* (d, J=1.5 Hz, 0.7H), 8.77 (d, J=1.5 Hz, 0.3H), 8.55-8.54 (m, 1H), 8.30* (dt, J=8.0, 1.8 Hz, 0.7H), 8.23* (s, 0.7H), 8.16 (d, J=8.0 Hz, 0.3H), 7.99 (s, 0.3H), 7.48 (dd, J=8.0, 5.0 Hz, 1.0H), 4.40 (t, J=5.0 Hz, 0.3H), 3.86 (dd, J=11.0, 4.5 Hz, 0.3H), 3.76-3.72 (m, 1.7H), 3.51* (t, J=5.5 Hz, 0.7H); ¹³C NMR (125 MHz, CD₃OD), described as mixture of (E/Z) isomers, a molar ratio ca. 7:3, with the signals of the major isomer marked with asterisks, δ 176.3, 172.9*, 151.4*, 151.1, 149.8*, 149.2, 146.5*, 142.5, 136.1*, 135.6, 132.3, 132.2*, 125.53, 125.50*, 65.5*, 65.1, 57.2*, 54.7; FIRMS (ESI, m/z): calcd for C₉H₁₃N₄O₂ ([M+H]⁺): 209.1033, Found: 209.1021.

### Synthesis and Characterization of (S)-2-Amino-3-hydroxy-N′-(pyridin-4-ylmethylene)-propanehydrazide, JYX049

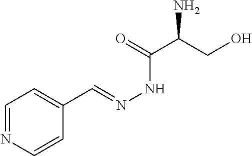 [SMILES: N[C@@H](CO)C(=O)N/N=C/c1ccncc1]

This product was prepared according to general procedure C. ¹H NMR (500 MHz, CD₃OD), described as mixture of (E/Z) isomers, a molar ratio ca. 7:3, signals of major isomer marked with asterisk, δ 8.57 (dd, J=5.0, 1.5 Hz, 2H), 8.20* (s, 0.7H), 7.95 (s, 0.3H), 7.78* (dd, J=4.5, 1.5 Hz, 1.4H), 7.68 (dd, J=4.5, 1.5 Hz, 0.6H), 4.44 (t, J=5.3 Hz, 0.3H), 3.88 (dd, J=11.5, 4.0 Hz, 0.3H), 3.78-3.74 (m, 1.7H), 3.54* (t, J=5.5 Hz, 0.7H); ¹³C NMR (125 MHz, CD₃OD), described as mixture of (E/Z) isomers, a molar ratio ca. 7:3, with the signals of the major isomer marked with asterisks, δ 176.0, 173.1*, 150.8*, 150.7*, 146.8*, 144.0, 142.9, 123.1*, 122.6, 65.4*, 64.8, 57.2*, 54.8; HRMS (ESI, m/z): calcd for C₉H₁₃N₄O₂ ([M+H]⁺): 209.1033, Found: 209.1029.

### Synthesis and Characterization of (S)-2-Amino-3-hydroxy-N′-((4-oxo-1,4-dihydropyridin-3-yl)methylene)propanehydrazide, JYX050

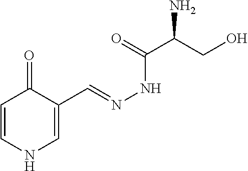 [SMILES: N[C@@H](CO)C(=O)N/N=C/c1c[nH]ccc1=O]

This product was prepared according to general procedure C. 1B NMR (500 MHz, d₆-DMSO), described as mixture of (E/Z) isomers, a molar ratio ca. 7:3, with the signals of the major isomer marked with asterisks, δ 8.37* (s, 0.7H), 8.12-8.11 (m, 1H), 8.09 (s, 0.3H), 7.68-7.65 (m, 1H), 6.22-6.19 (m, 1H), 4.10 (t, J=5.5 Hz, 0.3H), 3.57 (dd, J=10.5, 4.5 Hz, 0.3H), 3.50-3.40 (m, 1.7H), 3.25* (t, J=5.8 Hz, 0.7H); ¹³C NMR (125 MHz, d₆-DMSO), described as mixture of (E/Z) isomers, a molar ratio ca. 7:3, with the signals of the major isomer marked with asterisks, δ 175.1, 174.8, 174.0, 169.4*, 142.1*, 138.5*, 138.1, 137.6, 135.3, 134.5, 121.2*, 120.9*, 117.0*, 116.9*, 63.9*, 63.5, 55.8*, 52.8; HRMS (ESI, m/z): calcd for C₉H₁₃N₄O₃ ([M+H]⁺): 225.0982, Found: 225.0976.

### Synthesis and Characterization of N-Methoxy-2-(2-(2,3,4-trihydroxyphenyl)acetamido)acetamide, JYX051

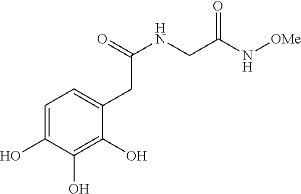

This product was prepared according to general procedure F. ¹H NMR (500 MHz, CD₃OD) δ 6.49 (d, J=8.0 Hz, 1H), 6.32 (d, J=8.0 Hz, 1H), 3.76 (s, 2H), 3.67 (s, 3H), 3.48 (s, 2H); ¹³C NMR (125 MHz, CD₃OD) δ 176.0, 168.5, 146.6, 145.5, 134.9, 121.8, 115.0, 108.2, 64.4, 41.7, 38.8; HRMS (ESI, m/z): calcd for C₁₁H₁₃N₂O₆ ([M−H]⁻): 269.0779, Found: 269.078.

### Example 4: ENPP1 Inhibition Assay

To identify small molecule inhibitors of ENPP1, a cell free luciferase based luminescent assay was established. Test compounds were dispensed by automated workstation (Beckman, Biomek FX) in 384 well plates (Corning, 4513). Every 384 well plate included 32 wells each for negative and for positive internal controls. Negative controls included human recombinant ENPP1 protein and ATP while positive controls included ATP without ENPP1. Human ENPP1 recombinant protein was added to all experimental wells with an automated washer dispenser (BioTek, EL406) and incubated at room temperature. After 30 minutes, ATP was added to all the wells in the plate by washer dispenser and incubated at room temperature for 120 minutes. Cell titer-Glo was then added to whole plate and incubated for 10 minutes. The degree of luminescence in each well were detected by plate reader (PerkinElmer, 2105) and the results are shown in Table 1 as a percent of the positive control, which included no ENPP1.

TABLE 1

| | degree of | | degree of |
|---|---|---|---|
| | luminescence as | | luminescence as |
| | a percent of | | a percent of |
| Compound | positive control | Compound | positive control |
| JYX001 | + | JHD009 | +++ |
| JYX002 | + | JHD019 | ++ |
| JYX003 | + | JHD030 | + |
| JYX004 | + | JHD034 | + |
| JYX005 | + | JHD035 | + |
| JYX007 | + | JHD036 | ++ |
| JYX008 | + | JHD037 | + |
| JYX009 | + | JHD038 | ++ |
| JYX010 | + | JHD039 | + |
| JYX011 | + | JHD040 | ++ |
| JYX012 | + | JHD044 | +++ |
| JYX013 | + | JHD045 | ++ |
| JYX014 | + | JHD046 | + |
| JYX015 | + | JHD047 | ++ |
| JYX016 | + | JHD048 | + |
| JYX017 | + | JHD051 | ++ |
| JYX018 | + | | |
| JYX019 | ++ | | |
| JYX020 | ++ | | |
| JYX021 | + | | |
| JYX022 | +++ | | |
| JYX023 | + | | |
| JYX024 | + | | |
| JYX025 | + | | |
| JYX026 | + | | |
| JYX027 | + | | |
| JYX028 | + | | |
| JYX029 | + | | |
| JYX030 | +++ | | |
| JYX031 | ++ | | |
| JYX032 | +++ | | |
| JYX033 | ++ | | |
| JYX034 | ++ | | |
| JYX035 | +++ | | |
| JYX036 | +++ | | |
| JYX037 | +++ | | |
| JYX038 | +++ | | |
| JYX039 | +++ | | |
| JYX040 | + | | |
| JYX041 | ++ | | |
| JYX042 | + | | |
| JYX043 | + | | |
| JYX044 | +++ | | |
| JYX045 | + | | |
| JYX046 | + | | |
| JYX047 | + | | |
| JYX048 | + | | |
| JYX049 | + | | |
| JYX050 | + | | |
| JYX051 | +++ | | |

+++ means ≥30% of positive control; ++ means between 10% and 30% of positive control; + means ≤10% positive control.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound of Formula I, II, III, or IV: 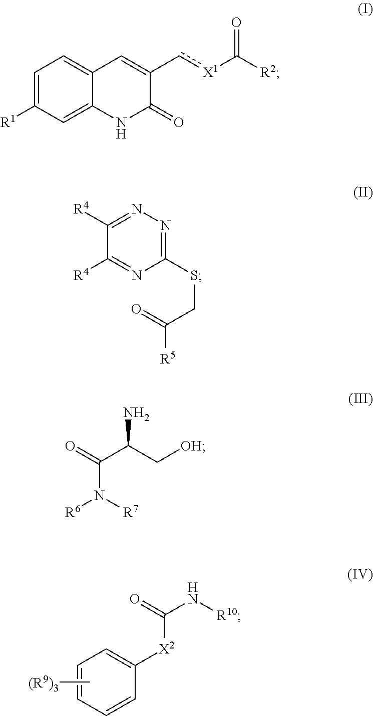
or a pharmaceutically acceptable salt thereof;
wherein:
R¹ is selected from —OH, alkoxy, or halo;
R² is selected from alkyl, aryl, or heteroaryl;
X¹ is —N(R³)— and [symbol] is a single bond, or ═C(H)— and [symbol] is a double bond;
R³ is aralkyl or heteroaralkyl;
R⁴ is heteroaryl;
R⁵ is selected from arylamine or aryl;
R⁶ is H;
R⁷ is selected from 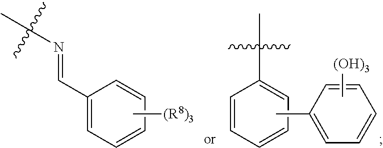
or R⁶ and R⁷, together with the N to which they are attached, form 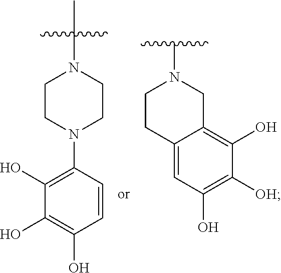
and
R⁸ is, independently for each occurrence, a non-hydrogen substituent;
R⁹ is, independently for each occurrence, a non-hydrogen substituent;
X² is selected from alkylene, alkylene-NH—, or heteroarylene; and
R¹⁰ is selected from -alkylene-C(O)NHOH, -alkylene-C(O)NH-alkoxy, -alkylene-C(O)NH-alkyl, aryl, or -alkylene-aryl, wherein aryl is optionally substituted.

2. The compound of claim 1, wherein the compound is a compound of Formula I: 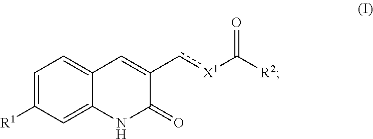
or a pharmaceutically acceptable salt thereof.

3. The compound of claim 1 or 2, wherein:
R¹ is selected from —OH, —O—C₁₋₄ alkyl, or halo;
R² is selected from C₁₋₄ alkyl, monocyclic aryl, bicyclic aryl, monocyclic heteroaryl, or bicyclic heteroaryl, wherein the monocyclic aryl is optionally substituted with 1-3 substituents selected, independently for each occurrence, from —OH or —O—C₁₋₄ alkyl;
X¹ is —N(R³)— and [symbol] is a single bond or X is ═C(H)— and [symbol] is a double bond; and
R³ is heteroarylmethyl.

4. The compound of any one of claims 1-3, wherein R¹ is selected from —OH, —OMe, or halo.

5. The compound of any one of claims 1-4, wherein R² is selected from phenyl, naphthyl, benzothiazole, benzofuran, benzoxazole, pyridyl, or methyl, wherein phenyl is optionally substituted with 1-3 substituents selected, independently for each occurrence, from —OH or —OMe.

6. The compound of any one of claims 1-5, wherein R² is phenyl, optionally substituted with 1-3 substituents selected, independently for each occurrence, from —OH or C₁₋₄ alkoxy.

7. The compound of any one of claims 1-6, wherein R³ is thienylmethyl or furylmethyl.

8. The compound of any one of claims 1-7, wherein:
R¹ is selected from —OH, —OMe, or halo;
R² is selected from phenyl, naphthyl, benzothiazole, benzofuran, benzoxazole, pyridyl, or methyl, and is optionally substituted with 1-3 substituents selected, independently for each occurrence, from —OH or —OMe; and
R³ is thienylmethyl or furylmethyl.

9. The compound of any one of claims 1-8, wherein the compound is selected from: 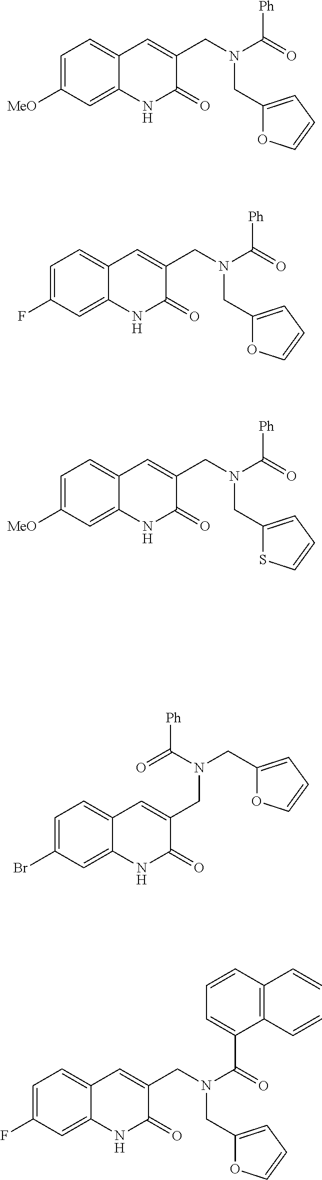 [SMILES: COc1ccc2cc(CN(Cc3ccco3)C(=O)c3ccccc3)c(=O)[nH]c2c1 ; O=C(c1ccccc1)N(Cc1ccco1)Cc1cc2ccc(F)cc2[nH]c1=O ; COc1ccc2cc(CN(Cc3cccs3)C(=O)c3ccccc3)c(=O)[nH]c2c1 ; O=C(c1ccccc1)N(Cc1ccco1)Cc1cc2ccc(Br)cc2[nH]c1=O ; C=Cc1cccc(C(=O)N(Cc2ccco2)Cc2cc3ccc(F)cc3[nH]c2=O)c1C=C] 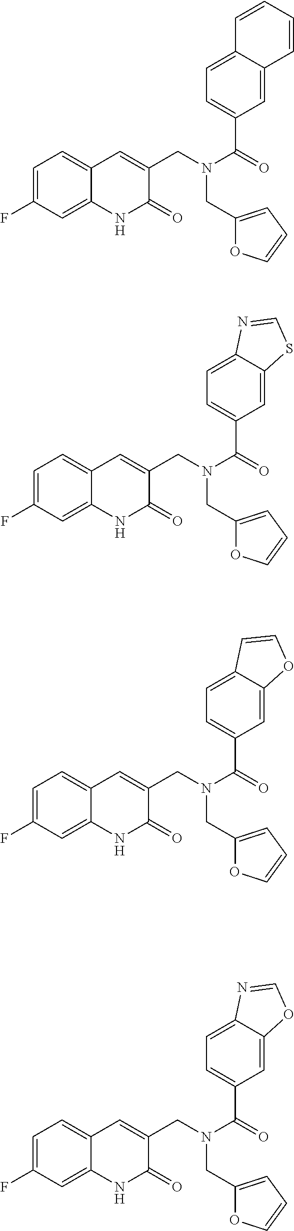 [SMILES: O=C(c1ccc2ccccc2c1)N(Cc1ccco1)Cc1cc2ccc(F)cc2[nH]c1=O ; O=C(c1ccc2ncsc2c1)N(Cc1ccco1)Cc1cc2ccc(F)cc2[nH]c1=O ; O=C(c1ccc2ccoc2c1)N(Cc1ccco1)Cc1cc2ccc(F)cc2[nH]c1=O ; O=C(c1ccc2ncoc2c1)N(Cc1ccco1)Cc1cc2ccc(F)cc2[nH]c1=O] 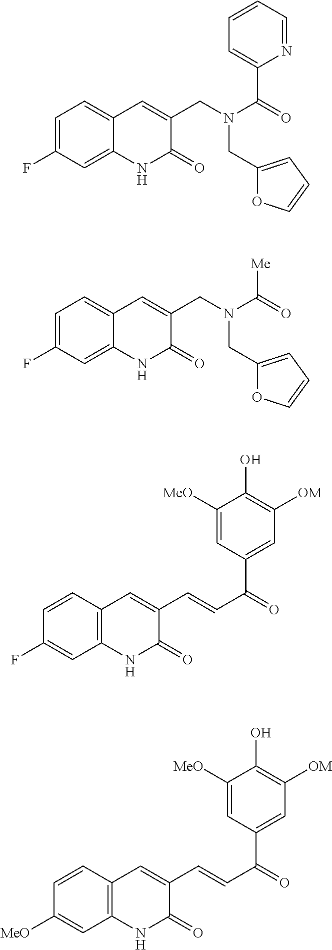
or a pharmaceutically acceptable salt thereof.

10. The compound of claim 1, wherein the compound is a compound of Formula II: 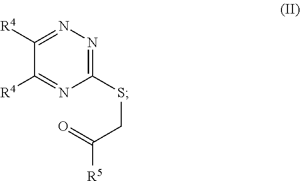
or a pharmaceutically acceptable salt thereof.

11. The compound of claim 1 or 10, wherein:
R⁴ is monocyclic heteroaryl; and
R⁵ is selected from —NH-monocyclic aryl or -monocyclic aryl, wherein the aryl is optionally substituted with 1-3 substituents selected, independently for each occurrence, from halo, —NO₂, —OH, C₁₋₄ alkoxy, —NH₂, or —NHAc.

12. The compound of any one of claim 1, 10, or 11, wherein R⁴ is furyl or thienyl.

13. The compound of any one of claim 1 or 10-12, wherein R⁵ is —NH-phenyl or phenyl, wherein the phenyl is optionally substituted with 1-3 substituents selected, independently for each occurrence, from halo, —NO₂, —OH, —OMe, —NH₂, or —NHAc.

14. The compound of any one of claim 1 or 10-13, wherein:
R⁴ is furyl or thienyl; and
R⁵ is —NH-phenyl or phenyl, wherein the phenyl is optionally substituted with 1-3 substituents selected, independently for each occurrence, from halo, —NO₂, —OH, —OMe, —NH₂, or —NHAc.

15. The compound of any one of claim 1 or 10-14, wherein the compound is selected from: 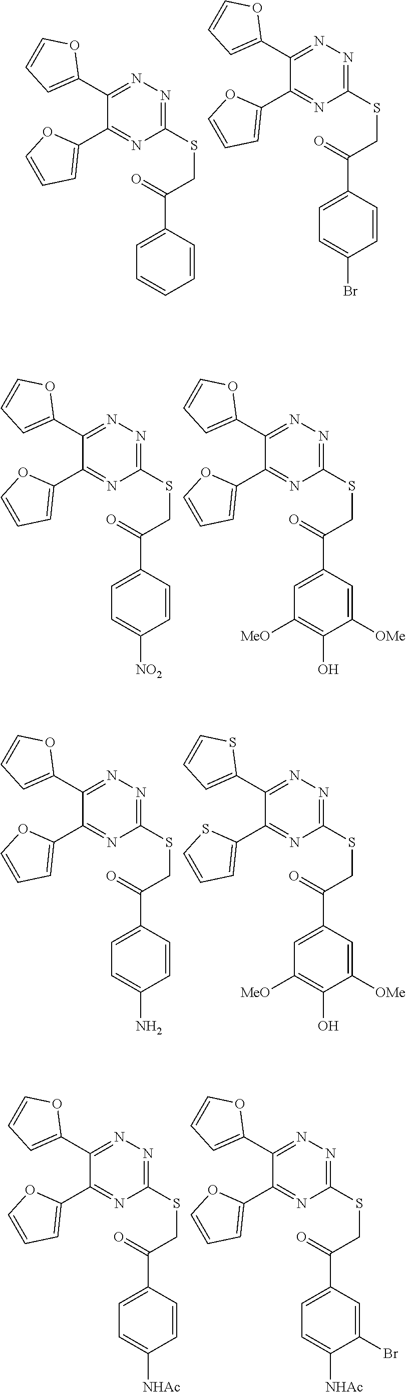 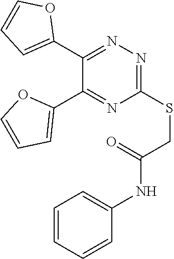 [SMILES: O=C(CSc1nnc(-c2ccco2)c(-c2ccco2)n1)Nc1ccccc1]
or a pharmaceutically acceptable salt thereof.

16. The compound of claim 1, wherein the compound is a compound of Formula III: 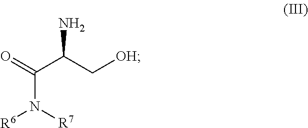
or a pharmaceutically acceptable salt thereof.

17. The compound of claim 16, wherein R⁸ is, independently for each occurrence, selected from —OH, —NHAc, halo, or hydroxyalkyl.

18. The compound of claim 17, wherein R⁸ is, independently for each occurrence, selected from —OH, —NHAc, halo, or C₁₋₄ hydroxyalkyl.

19. The compound of any one of claims 17-18, wherein R⁸ is, independently for each occurrence, selected from —OH, —NHAc, halo, or —CH₂OH.

20. The compound of any one of claims 16-19, wherein:
R⁶ is H;
R⁷ is selected from 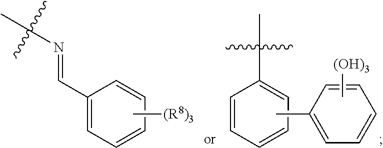
or R⁶ and R⁷, together with the N to which they are attached, together form 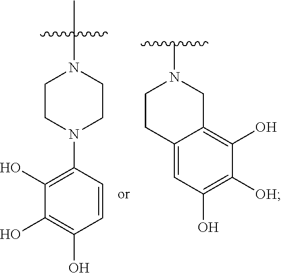
and
R⁸ is, independently for each occurrence, selected from —OH, —NHAc, halo, or —CH₂OH.

21. The compound of claim 20, wherein the compound is selected from: 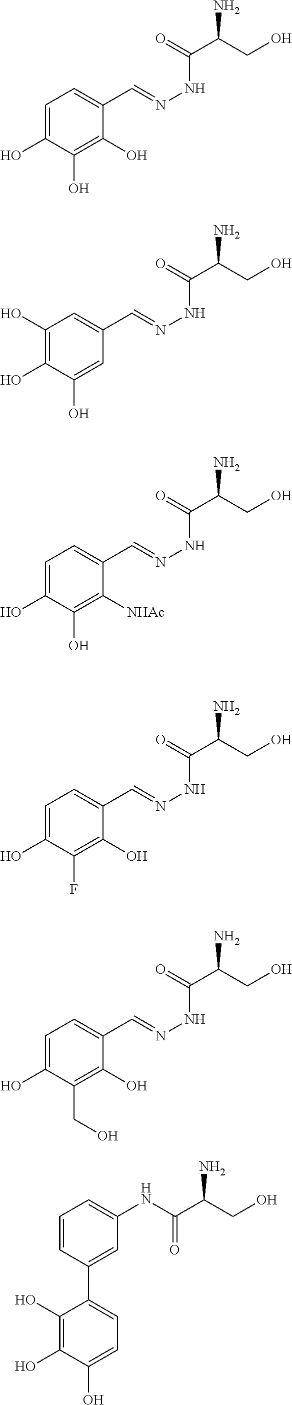 [SMILES: N[C@@H](CO)C(=O)N/N=C/c1ccc(O)c(O)c1O ; N[C@@H](CO)C(=O)N/N=C/c1cc(O)c(O)c(O)c1 ; CC(=O)Nc1c(/C=N/NC(=O)[C@@H](N)CO)ccc(O)c1O ; N[C@@H](CO)C(=O)N/N=C/c1ccc(O)c(F)c1O ; N[C@@H](CO)C(=O)N/N=C/c1ccc(O)c(CO)c1O ; N[C@@H](CO)C(=O)Nc1cccc(-c2ccc(O)c(O)c2O)c1] 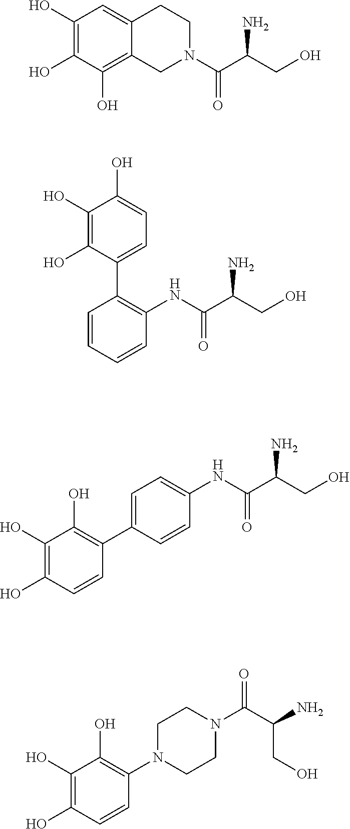 [SMILES: N[C@@H](CO)C(=O)N1CCc2cc(O)c(O)c(O)c2C1 ; N[C@@H](CO)C(=O)Nc1ccccc1-c1ccc(O)c(O)c1O ; N[C@@H](CO)C(=O)Nc1ccc(-c2ccc(O)c(O)c2O)cc1 ; N[C@@H](CO)C(=O)N1CCN(c2ccc(O)c(O)c2O)CC1]
or a pharmaceutically acceptable salt thereof.

22. The compound of claim 1, wherein the compound is a compound of Formula IV: 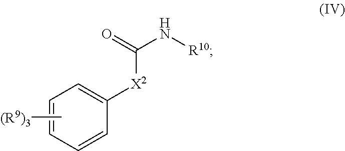
or a pharmaceutically acceptable salt thereof.

23. The compound of claim 22, wherein R⁹ is, independently for each occurrence, selected from —OH or alkoxy.

24. The compound of any one of claim 22 or 23, wherein R¹⁰ is selected from -alkylene-C(O)NHOH, -alkylene-C(O)NH-alkoxy, -alkylene-C(O)NH-alkyl, aryl, or -alkylene-aryl, wherein aryl is optionally substituted with 1-3 substituents selected, independently for each occurrence, from halo, —OH, or alkoxy.

25. The compound of any one of claims 22-24, wherein:
R⁹ is, independently for each occurrence, selected from —OH or —O—C₁₋₄ alkyl;
X² is selected from monocyclic heteroarylene, —C₁₋₄ alkylene-, or —C₁₋₄ alkylene-NH—, wherein the NH is bonded to the carbonyl; and
R¹⁰ is selected from —C₁₋₄ alkylene-C(O)NHOH, —C₁₋₄ alkylene-C(O)NH—C₁₋₄ alkyl, monocyclic aryl, or —C₁₋₄ alkylene-monocyclic aryl, wherein each monocyclic aryl is optionally substituted with 1-3 substituents selected, independently for each occurrence, from halo, —OH, or —O—C₁₋₄ alkyl.

26. The compound of any one of claims 22-25, wherein R⁹ is, independently for each occurrence, selected from —OH or —OMe.

27. The compound of any one of claims 22-26, wherein X² is selected from —CH₂—, —CH₂CH₂NH—, or pyrazolyl.

28. The compound of any one of claims 22-27, wherein R¹⁰ is selected from —CH₂C(O)NHOH, —CH₂C(O)NHMe, phenyl, or —CH₂-phenyl, wherein phenyl is optionally substituted with 1-3 substituents selected, independently for each occurrence, from halo, —OH, or —OMe.

29. The compound any one of claims 22-28, wherein:
R⁹ is, independently for each occurrence, selected from —OH or —OMe;
X² is selected from —CH₂—, —CH₂CH₂NH—, or pyrazolyl; and
R¹⁰ is selected from —CH₂C(O)NHOH, —CH₂C(O)NHMe, phenyl, or —CH₂-phenyl, wherein phenyl is optionally substituted with 1-3 substituents selected, independently for each occurrence, from halo, —OH, or —OMe.

30. The compound of any one of claims 22-29, wherein the compound is selected from: 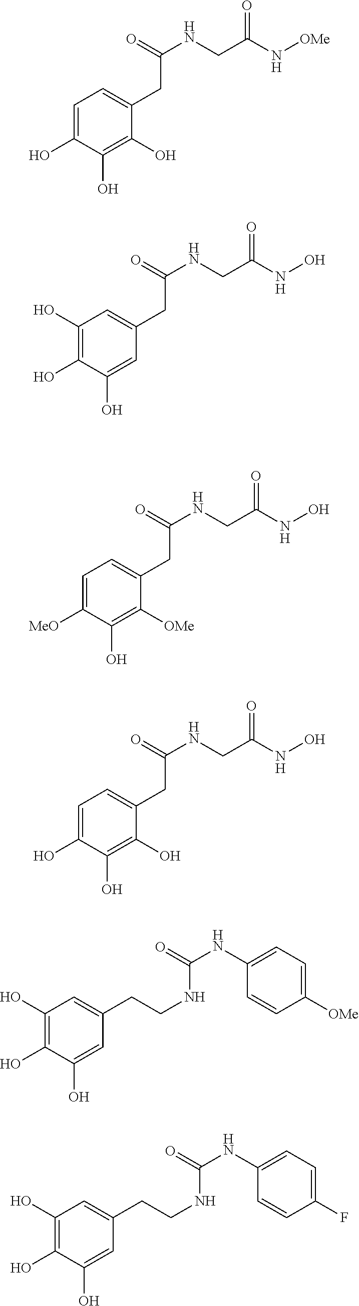 [SMILES: CONC(=O)CNC(=O)Cc1ccc(O)c(O)c1O ; O=C(CNC(=O)Cc1cc(O)c(O)c(O)c1)NO ; COc1ccc(CC(=O)NCC(=O)NO)c(OC)c1O ; O=C(CNC(=O)Cc1ccc(O)c(O)c1O)NO ; COc1ccc(NC(=O)NCCc2cc(O)c(O)c(O)c2)cc1 ; O=C(NCCc1cc(O)c(O)c(O)c1)Nc1ccc(F)cc1] 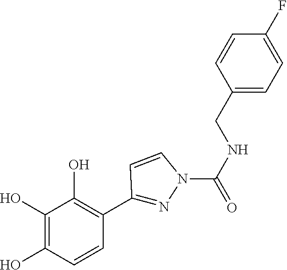 [SMILES: O=C(NCc1ccc(F)cc1)n1ccc(-c2ccc(O)c(O)c2O)n1]
or a pharmaceutically acceptable salt thereof.

31. A pharmaceutical composition comprising the compound of any one of claims 1-30, or a pharmaceutically acceptable salt thereof, together with one or more pharmaceutically acceptable excipients, diluents, or carriers.

32. A method of treating myocardial infarction in a subject in need thereof, the method comprising administering to the subject a therapeutically acceptable amount of the compound of any one of claims 1-30, or a pharmaceutically acceptable salt thereof, or the pharmaceutical composition of claim 31.

33. A method of preventing heart failure in a subject in need thereof, the method comprising administering to the subject a therapeutically acceptable amount of the compound of any one of claims 1-30, or a pharmaceutically acceptable salt thereof, or the pharmaceutical composition of claim 31.

34. A method of promoting cardiac wound healing in a subject in need thereof, the method comprising administering to the subject a therapeutically acceptable amount of the compound of any one of claims 1-30, or a pharmaceutically acceptable salt thereof, or the pharmaceutical composition of claim 31.

35. A method of preventing ectopic calcification of cardiac tissue in a subject in need thereof, the method comprising administering to the subject a therapeutically acceptable amount of the compound of any one of claims 1-30, or a pharmaceutically acceptable salt thereof, or the pharmaceutical composition of claim 31.

36. A method of preventing scarring of cardiac tissue in a subject in need thereof, the method comprising administering to the subject a therapeutically acceptable amount of the compound of any one of claims 1-30, or a pharmaceutically acceptable salt thereof, or the pharmaceutical composition of claim 31.

37. A method of preventing dilated cardiomyopathy in a subject in need thereof, the method comprising administering to the subject a therapeutically acceptable amount of the compound of any one of claims 1-30, or a pharmaceutically acceptable salt thereof, or the pharmaceutical composition of claim 31.

38. A method of enhancing cardiac repair in a subject in need thereof, the method comprising administering to the subject a therapeutically acceptable amount of the compound of any one of claims 1-30, or a pharmaceutically acceptable salt thereof, or the pharmaceutical composition of claim 31.

39. A method of preventing cell death of cardiac cells in a subject in need thereof, the method comprising administering to the subject a therapeutically acceptable amount of the compound of any one of claims 1-30, or a pharmaceutically acceptable salt thereof, or the pharmaceutical composition of claim 31.

40. A method of preventing release of one or more pro-inflammatory molecules from cardiac myocytes in a subject in need thereof, the method comprising administering to the subject a therapeutically acceptable amount of the compound of any one of claims 1-30, or a pharmaceutically acceptable salt thereof, or the pharmaceutical composition of claim 31.

41. A method of inhibiting ENPP1 activity in a subject in need thereof, the method comprising administering to the subject a therapeutically acceptable amount of the compound of any one of claims 1-30, or a pharmaceutically acceptable salt thereof, or the pharmaceutical composition of claim 31.

42. The compound of any one of claims 1-30, or a pharmaceutically acceptable salt thereof, or the pharmaceutical composition of claim 31 for use in treating myocardial infarction in a subject in need thereof.

43. The compound of any one of claims 1-30, or a pharmaceutically acceptable salt thereof, or the pharmaceutical composition of claim 31 for use in preventing heart failure in a subject in need thereof.

44. The compound of any one of claims 1-30, or a pharmaceutically acceptable salt thereof, or the pharmaceutical composition of claim 31 for use in promoting cardiac wound healing in a subject in need thereof.

45. The compound of any one of claims 1-30, or a pharmaceutically acceptable salt thereof, or the pharmaceutical composition of claim 31 for use in preventing ectopic calcification of cardiac tissue in a subject in need thereof.

46. The compound of any one of claims 1-30, or a pharmaceutically acceptable salt thereof, or the pharmaceutical composition of claim 31 for use in preventing scarring of cardiac tissue in a subject in need thereof.

47. The compound of any one of claims 1-30, or a pharmaceutically acceptable salt thereof, or the pharmaceutical composition of claim 31 for use in preventing dilated cardiomyopathy in a subject in need thereof.

48. The compound of any one of claims 1-30, or a pharmaceutically acceptable salt thereof, or the pharmaceutical composition of claim 31 for use in enhancing cardiac repair in a subject in need thereof.

49. The compound of any one of claims 1-30, or a pharmaceutically acceptable salt thereof, or the pharmaceutical composition of claim 31 for use in preventing cell death of cardiac cells in a subject in need thereof.

50. The compound of any one of claims 1-30, or a pharmaceutically acceptable salt thereof, or the pharmaceutical composition of claim 31 for use in preventing release of one or more pro-inflammatory molecules from cardiac myocytes in a subject in need thereof.

51. The compound of any one of claims 1-30, or a pharmaceutically acceptable salt thereof, or the pharmaceutical composition of claim 31 for use in inhibiting ENPP1 activity in a subject in need thereof.
